SEQUENCE LISTING (15 sequences)

SEQ ID NO: 1 - protein, 37 aa, Artificial Sequence
  Description of Artificial Sequence Synthetic polypeptide
YKRWIILGLNKIVRMYSQQEKNEQELLELDKWASLWN

SEQ ID NO: 2 - protein, 37 aa, Artificial Sequence
  Description of Artificial Sequence Synthetic polypeptide
QQEKNEQELLELDKWASLWNYKRWIILGLNKIVRMYS

SEQ ID NO: 3 - protein, 33 aa, Artificial Sequence
  Description of Artificial Sequence Synthetic polypeptide
YKRWIILGLNKIVRMYSSLWNWFNITNWLWYIK

SEQ ID NO: 4 - protein, 33 aa, Artificial Sequence
  Description of Artificial Sequence Synthetic polypeptide
SLWNWFNITNWLWYIKYKRWIILGLNKIVRMYS

SEQ ID NO: 5 - protein, 852 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAERRVVEREERAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 6 - protein, 854 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAERRVVEREERAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALLKK

SEQ ID NO: 7 - protein, 852 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALL

SEQ ID NO: 8 - protein, 854 aa, Artificial Sequence
  Description of Artificial Sequence Synthetic polypeptide
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLQLTVWGIKQLQARVLAVERYLKDQQLLGIWGCSGKLICTTTVPWNSSWSNKSQDEIWDNMTWMEWEREINNYTDIIYSLIEESQNQQEKNEQELLALDKWASLWNWFDITNWLWYIKIFIMIVGGLIGLRIVFAVLSIVNRVRQGYSPLSFQTLIPNPRGPDRPEGIEEEGGEQDRDRSIRLVNGFLALAWDDLRSLCLFSYHRLRDFILIAARTVELLGRKGLRRGWEALKYLWNLLQYWGQELKNSAISLLDTTAIAVAEGTDRVIEVVQRACRAILNIPRRIRQGLERALLKK

SEQ ID NO: 9 - protein, 847 aa, Artificial Sequence
  Description of Artificial Sequence Synthetic polypeptide
MRVKGIRKNYQHLWRGGTLLLGIIVICSAVEKLWVTVYYGVPVWKEATTTLFCASDAKAYDTEVHNVWATHACVPTDPNPQEVVLGNVTEKFNMWKNNMVEQMQEDIISLWDQSLKPCVKLTPLCVTLNCKDVNATNTTNGSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMKDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLIGLRIVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 10 - protein, 5 aa, Artificial Sequence
  Description of Artificial Sequence Synthetic peptide
AVERY

SEQ ID NO: 11 - protein, 849 aa, Artificial Sequence
  Description of Artificial Sequence Synthetic polypeptide
MRVKGIRKNYQHLWRGGTLLLGIIVICSAVEKLWVTVYYGVPVWKEATTTLFCASDAKAYDTEVHNVWATHACVPTDPNPQEVVLGNVTEKFNMWKNNMVEQMQEDIISLWDQSLKPCVKLTPLCVTLNCKDVNATNTTNGSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMKDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLIGLRIVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALLKK

SEQ ID NO: 12 - protein, 847 aa, Artificial Sequence
  Description of Artificial Sequence Synthetic polypeptide
MRVKGIRKNYQHLWRGGTLLLGIIVICSAVEKLWVTVYYGVPVWKEATTTLFCASDAKAYDTEVHNVWATHACVPTDPNPQEVVLGNVTEKFNMWKNNMVEQMQEDIISLWDQSLKPCVKLTPLCVTLNCKDVNATNTTNGSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMKDNWRSELYKYKVVKIEPLGVAPTKAERRVVQREERAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLIGLRIVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 13 - protein, 849 aa, Artificial Sequence
  Description of Artificial Sequence Synthetic polypeptide
MRVKGIRKNYQHLWRGGTLLLGIIVICSAVEKLWVTVYYGVPVWKEATTTLFCASDAKAYDTEVHNVWATHACVPTDPNPQEVVLGNVTEKFNMWKNNMVEQMQEDIISLWDQSLKPCVKLTPLCVTLNCKDVNATNTTNGSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMKDNWRSELYKYKVVKIEPLGVAPTKAERRVVQREERAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLIGLRIVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALLKK

SEQ ID NO: 14 - protein, 376 aa, Artificial Sequence
  Description of Artificial Sequence Synthetic polypeptide
MRVRGIQRNCQHLWRWGTLILGMLMICSAARAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 15 - protein, 378 aa, Artificial Sequence
  Description of Artificial Sequence Synthetic polypeptide
MRVRGIQRNCQHLWRWGTLILGMLMICSAARAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALLKK

(19) United States
(12) Patent Application Publication
(10) Pub. No.: US 2010/0285106 A1
(43) Pub. Date: Nov. 11, 2010

(54) MULTICOMPONENT VACCINE

(76) Inventors: Barton F. Haynes, Durham, NC (US); Nancy G. Smith, Durham, NC (US); S.Munir Alam, Durham, NC (US); Feng Gao, Durham, NC (US); Hua-Xin Liao, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/312,587

(22) PCT Filed: Nov. 19, 2007
(86) PCT No.: PCT/US07/24122

§ 371 Date: May 21, 2010

Related U.S. Application Data
(60) Provisional application No. 60/859,496, filed on Nov. 17, 2006.

(51) Int. Cl. A61K 39/21; A61K 9/127; A61K 39/395; A61P 31/18; A61P 37/04

(52) U.S. Cl. 424/450; 424/130.1; 424/144.1; 424/160.1; 424/208.1

(57) ABSTRACT

The present invention relates, in general, to human immunodeficiency virus (HIV) and, in particular, to a multicomponent vaccine and method of using same to protect against HTV-I infection.

This application claims priority from U.S. Provisional Application No. 60/859,496. filed Nov. 17, 2006, the entire content of which is incorporated herein by reference.

This invention was made with government support under Grant No. AI0678501 awarded by the National Institutes of Health. The government has certain rights in the invention.

### TECHNICAL FIELD

The present invention relates, in general, to human immunodeficiency virus (HIV) and, in particular, to a multicomponent vaccine and method of using same to protect against HIV-1 infection.

### BACKGROUND

Production of an effective vaccine for HIV-1 is a critical goal of AIDS research. To date, development of a preventive vaccine has been unsuccessful due to the diversity of HIV (Gaschen, Science 296:2354 (2002)), the rapid onset of apoptosis of immune cells at mucosal sites (Mattapallil et al, Nature 434:1093 (2005); Veazey et al, Science 280:427 (1998); Guadalupe et al, J. Virol 77:11708 (2003); Brenchley et al, J. Exp. Med. 200:749 (2004); Menhandru et al, J. Exp. Med. 200:761 (2004)), the fact that HIV-1 is an integrating virus with a viral cellular reservoir (Fauci, Science 245:305 (1989)), and the delay in induction of autologous HIV-1 innate and neutralizing antibody responses from eight weeks to a year following viral ramp-up in the plasma (Abel et al, J. Virol 80:6357-67 (2006), Wei et al, Nature 422:307-12 (2003); Richman et al, Proc. Natl. Acad. Sci. USA 100:4144-9 (2003)).

The present invention relates to a multicomponent vaccine that addresses problems resulting from the diversity of HIV by the use consensus and/or mosaic HIV genes (Gaschen et al, Science 296:2354 (2002); Liao et al, Virology 353:268 (2006), Gao et al, J. Virol. 79:1154 (2005), Weaver et al, J. Virol. 80:6754 (2006), Fischer et al, Nature Medicine, 13(1):100-106 (2007), Epub 2006 Dec. 24), coupled with strategies designed to break immune tolerance to allow for induction of the desired specificity of neutralzing antibodies at mucosal sites (e.g., through the use of T regulatory cell inhibition and/or TLR-9 agonist adjuvants), and strategies designed to overcome HIV-1 induced apoptosis (e.g., induction of anti-phosphatidylserine (PS) antibodies, anti-CD36 antibodies, and/or anti-tat antibodies).

### SUMMARY OF THE INVENTION

The present invention relates generally to HIV. More specifically, the invention relates to a multicomponent HIV vaccine that can be used to protect humans against HIV-1 infection.

Objects and advantages of the present invention will be clear from the description that follows.

### BRIEF DESCRIPTION OF THE DRAWINGS

FIG. 1. Summary of antibody responses immediately following acute HIV-1 infection.

FIGS. 2A-2F. Fas ligand vs. viral load. (FIG. 2A) Fas ligand, panel 6246. (FIG. 2B) Fas ligand, panel 6240. (FIG. 2C) Fas ligand, panel 9076. (FIG. 2D) Fas ligand, panel 9021. (FIG. 2E) Fas ligand, panel 9020. (FIG. 2F) Fas ligand, panel 9032.

FIGS. 3A-3F. Fas (CD95) vs. viral load. (FIG. 3A) Fas (CD95), panel 6246. (FIG. 3B) Fas (CD95), panel 6240. (FIG. 3C) Fas (CD95), panel 9076. (FIG. 3D) Fas (CD95), panel 9021. (FIG. 3E) Fas (CD95), panel 9020. (FIG. 3F) Fas (CD95), panel 9032.

FIGS. 4A-4E. TNFR2 vs. viral load. (FIG. 4A) TNFR2, panel 6240. (FIG. 4B) TNFR2. panel 6244. (FIG. 4C) TNFR2, panel 6246. (FIG. 4D) TNFR2, panel 9020. (FIG. 4E) TNFR2, panel 9021.

FIGS. 5A and 5B. TRAIL (TNF-Related Apoptosis Inducing Ligand). (FIG. 5A) TRAIL, panel 9020. (FIG. 5B) TRAIL, panel 9021.

FIGS. 6A and 6B. PD-1 is upregulated on T and B cells in chronic HIV-1 infection. (FIG. 6A) CD3+. (FIG. 6B) CD19+.

FIGS. 7A-7D. (FIGS. 7A and 7B) Anti-PS on uninfected cells. (FIGS. 7C and 7D) Anti-PS on MN infected cells and virions.

FIGS. 8A and 8B. Binding of mAbs 4E10 and 2F5 to peptide-liposome conjugates. About 1000 RU of either synthetic liposomes (red); lipid-GTH1-4E10 (FIG. 8A, green); or 4E10-GTH1-lipid (FIG. 8A, blue) were anchored on to a BIAcore L1 sensor chip. A fourth flow cell was left untreated (magenta) with no lipid. On a second sensor chip, lipid-GTH1-2F5 (FIG. 8B, green); or 2F5-GTH1-lipid (FIG. 8B, blue) or liposomes alone (FIG. 8B, red) were anchored. Mab 4E10 (FIG. 8A) or mAb 2F5 (FIG. 8B) was injected over each sensor chip and the binding responses were recorded on a BIAcore 3000 instrument. The Kd values were derived from curve fitting analysis using the 2-step conformational change model and the BIAevalution software.

Methods. Phospholipids POPC (1-Palmitoyl-2-Oleoyl-sn-Glycero-3-Phosphatidylcholine), POPE (1-Palmitoyl-2-Oleoyl-sn-Glycero-3-Phosphatidylethanolamine), DOPE (1,2-Dioleoyl-sn-Glycero-3-Phosphatidylethanolamine); DMPA (1,2-Dimyristoyl-sn-Glycero-3-Phosphate) and Cholesterol dissolved in chloroform were purchased from Avanti Polar Lipids (Alabaster, Ala.). Phospholipid liposomes were prepared by dispensing appropriate molar amounts of phospholipids in chloroform resistant tubes. Chloroform solutions of lipids were added to the peptide solution, in molar ratios of 45:25:20:10 (POPC:POPE:DMPA:Cholesterol). HIV-1 membrane proximal peptides were dissolved in 70% Chloroform, 30% Methanol. Each peptide was added to a molar ratio of peptide:total phospholipids of 1:420. The phospholipids were mixed by gentle vortexing and the mixture was dried in the fume hood under a gentle stream of nitrogen. Any residual chloroform was removed by storing the lipids under a high vacuum (15 h). Aqueous suspensions of phospholipids were prepared by adding PBS or TBS buffer, pH 7.4 and kept at a temperature above the Tm for 10-30 minutes, with intermittent, vigorous vortexing to resuspend the phospholipids followed by Sonication in a bath sonicator (Misonix Sonicator 3000, Misonix Inc., Farmingdale, N.Y.). The sonicator was programmed to run 3 consecutive cycles of 45 seconds of total sonication per cycle. Each cycle included 5 seconds of sonication pulse (70 watts power output) followed by a pulse off period of 12 seconds. At the end of sonication, the suspension of lamellar liposomes was stored at 4° C. and was thawed and sonicated again as described above prior to capture on BLAcore sensor chip.

Peptides were synthesized and purified by reverse-phase HPLC and purity was confirmed by mass spectrometric analysis. Peptides used in this study include the following—

HIV-1 gp41 2F5 epitope peptides-
GTH1-2F5
(YKRWIILGLNKIVRMYS-QQEKNEQELLELDKWASLWN);
2F5-GTH1
(QQEKNEQELLELDKWASLWN-YKRWIILGLNKIVRMYS);
and
HIV-1 gp41 4E10 epitope peptides,
GTH1-4E10
(YKRWIILGLNKIVRMYS-SLWNWFNITNWLWYIK);
4E10-GTH1
(SLWNWFNITNWLWYIK-YKRWIILGLNKIVRMYS)

FIG. 9. Scheme of the detrimental acute infection events that the multicomponent vaccine of the invention overcomes.

FIG. 10. Non-human primate (NHP) ONTAK depletion (dose/kinetics).

FIG. 11. T-Regs in NHPs immunized with rPA.

FIG. 12. Anti-PA binding ELISA.

FIGS. 13A and 13B. Anthrax toxin neutralization.

FIGS. 14A-14C. Development of flow cytometric techniques for measurement of plasma apoptotic MP. In order to develop a novel protocol to assay the plasma with flow cytometry, a mixture of polystyrene beads was first assayed (FIG. 14A). Beads ranging from 0.1 μm to 1.0 μm in size were mixed in equal proportion, diluted, and analyzed with a BD LSRII. These sizes were used in accordance with previous studies defining microparticles by their size (Werner, Arterioscler. Thromb. Vasc. Biol. 26(1):112-6 (2006) Epub 2005 Oct. 20, Distler et al, Apoptosis 10:731-741 (2005)). Side scatter was used as a size discriminator because of the enhanced ability of the photomultiplier tube to discriminate smaller particles than the diode of the forward scatter detector. To determine optimal dilution ranges, a series of serial dilutions of the polystyrene bead mixture was analyzed (FIG. 14B). By performing such an experiment, it was discovered that any sample that is not dilute enough will yield an event count that is falsely low due to coincidence and high abort rates. An aborted event occurs-when the flow cytometer cannot process events because they arrive too close together or too fast for the system to process individually (coincidence). By diluting the sample to the point where only one particle flows through the detector at a time, the event count processed by the cytometer is more accurate. In fact, when the bead mixture was diluted at 1:1000, the 4 different sizes of beads could not be discriminated well, whereas clear populations of each size could be detected at a 1:100,000 dilution. To analyze plasma microparticles, (FIG. 14C), similar dilution series were used to experimentally determine the optimal dilution. (data not shown). To eliminate the possibility of counting debris that is present in plasma, but is smaller than the cellular microparticles and does not have forward or side scatter, the events occurring within a defined microparticle gate were counted. This gate was drawn by including the 0.1 μm beads in the low side scatter range, and including the 1.0 μm beads in the higher side scatter range, while excluding particles that had very little forward and side scatter, (red boxes in FIGS. 14A and 14C). The polystyrene sizing beads were run at a 1:100,000 dilution for each and every experimental run, allowing all data to be gated in the same manner. In plasma samples, it was found that the majority of the microparticles were between 0.1 and 0.5 μm, (the population within the red microparticle gate that demonstrated side scatter area of less than 10⁴). Larger microparticles, greater than 0.5 μm but smaller than 1.0 μm, were present but were fewer in proportion.

FIGS. 15A-15D. The effects of freeze/thaw cycles on the phenotype of plasma MP. Due to the low expression levels of some of the extracellular markers in the plasma donor samples, an investigation was made of the effects of freezing and thawing the plasma on the phenotype of the microparticles. Plasma from a chronically infected donor was divided into 3 aliquots. The first remained at 20° C. (fresh). The second was frozen for 10 minutes at −80° C. and thawed (frozen 1×), and the third was frozen similarly, thawed, and re-frozen, (frozen 2×). All three samples were then diluted, filtered, and centrifuged. The MP resuspension was stained with CD3 (FIG. 15A), CD45 (FIG. 15B), CD61 (a platelet MP marker) (FIG. 15C), and Annexin V (FIG. 15 D). The percentages within the green boxes indicate the percentage of MP positive for that particular marker after background subtraction of the isotype controls assayed simultaneously. These percentages were observed to increase upon the first freeze/thaw cycle and decrease after another freeze/thaw cycle, indicating that sample integrity plays an important role in the phenotyping of plasma MP.

FIGS. 16A-16C. Plasma viral loads of HIV, Hepatitis C Virus, (HCV) and Hepatitis B Virus (HBV) subjects. Thirty HIV+ seroconversion plasma panels (HBV and HCV negative), ten HBV seroconversion panels (HIV negative), and 10 HCV seroconversion panels (HIV negative) were studied. Panels demonstrate the kinetics of viral load ramp-up in HIV (FIG. 16A), HCV (FIG. 16B), and HBV (FIG. 16C). Day 0 was determined to be the first day that the viral load reached 100 copies/ml for HIV, 600 copies/ml for HCV, and 700 copies/ml for HBV.

FIGS. 17A-17C. Plasma markers of apoptosis. FIG. 17A. TRAIL, TNFR2, and Fas Ligand were measured for each plasma sample by ELISA and compared to viral load levels. Three representative subjects are shown. FIG. 17B. In order to compare increases in plasma markers of apoptosis between subjects, the mean before day 0 was compared to the mean after day 0, and percent increases were calculated. FIG. 17C. The same plasma markers of apoptosis were measured in HCV and HBV infected subjects. The results of one HCV and one HBV subject are shown.

FIGS. 18A and 18B. Summary of plasma markers of apoptosis. FIG. 18A. Boxplot analyses were performed for each group of data. The results of the acute HIV-1, HBV and HCV panels are displayed, with vertical lines signifying the maximum and minimum values. The P values were computed with a Student's T test. Blue boxes indicate p<0.01. FIG. 18B. Timing of peak analyte relative to maximum viral expansion. (10). Results are from a paired Wilcoxon rank test, and a low p value indicates that the two means (of the peak dates of interest) are significantly different. This implies that the mean ‘arrival times’ of the peaks (e.g., peak expansion day and peak TRAIL day) are significantly different. The ‘delay’ between the arrival times can be described in terms of a mean, a median, and an interquartile range. On this panel the ‘arrival time’ of each analyte maximum is compared with the time of peak viral expansion (red box). A p value arising from the Wilcoxon test is shown above the analyte of interest. Also noted are mean delay times (median times in parentheses). Open circles indicate outlier values.

FIGS. 19A and 19B. Relative microparticle counts in plasma samples. FIG. 19A. For each of 30 subjects studied, relative microparticle counts were acquired for each sequential time point. Three representative subjects are shown. FIG. 19B. The same analysis was performed for 10 HBV and 10 HCV infected subjects. The results of one HCV and one HBV subject are shown.

FIG. 20. Transmission electron micrograph of plasma MP harvested from an acute HIV-1 infected subject. Plasma MP were pelleted by ultracentrifugation and purified over a sucrose pad. MP range 0.05 micron to 0.8 micron in size.

### DETAILED DESCRIPTION OF THE INVENTION

The present invention relates to a multicomponent, multifunctional HIV vaccine targeted at overcoming: i) HIV diversity, ii) tolerance constraints of neutralizing antibody induction, and iii) apoptotic induced immunosuppression. The invention provides an HIV vaccine comprising centralized HIV gene inserts (consensus, mosaic), a tolerance-breaking component (e.g. TLR-agonists, T regulatory cell innhibition), and a component that can inhibit the immunosuppression of apoptotsis, or inhibit apoptosis itself (e.g., anti-PS, anti-CD36 antibody induction, and/or anti-HIV tat antibody induction).

The use of adjuvants and other immunization regimens that result in antibody specificities being made that are not ordinarily made to HIV-1 envelope immunization have been proposed previously (PCT/US2006/013684; U.S. application Ser. No. 11/785,007; U.S. application Ser. No. 11/812,992; U.S. Prov. Application No. 60/960,413). This work derived from the observation that many of the broadly neutralizing anti-HIV-1 monoclonal antibodies are autoantibodies and are likely under immunoregulatory control (Haynes et al, Science 308:1906 (2005), Haynes et al, Human Antibodies 14:59 (2006)). One adjuvant regimen that has been used to break tolerance in mice is oligo CpGs in an oil-based adjuvant (Tran et al, Clin. Immunol. 109:278 (2003)). For humans, the B type of oligo CpGs can be used, including 2006 or 10103 oCpGs (McCluskie and Krieg, Curr. Topic. Microbial. Immunol. 311:155-178 (2006)). However, tolerance controls can be difficult to completely overcome, even on a temporary basis, and autoantibody production is also under T regulatory cell control (Shevach, Immunity 25:195-201 (2006)). Tnus, immunization with an adjuvant regimen combined with a regimen to temporarily inactivate T regulatory cells can be used to induce anti-HIV-1 antibodies that normally are prevented from being induced by negative immunoregulatory mechanisms. T regulatory cells can be inactivated or eliminated by either immunizing with glucocorticoid-induced TNT family-related receptor ligand (GITRL) DNA (Stone et al, J. Virol. 80:1762-72 (2006)), CD40 Ligand DNA (Stone et al, Clin. Vaccine Immunol. 13:1223-30 (2006), or administering simultaneously with the vaccine immunization a CD25 mab or ONTAK, a IL-2-toxin conjugate (see PCT/US2005/37384, PCT/US06/47591, U.S. application Ser. No. 11/302,505 and U.S. application Ser. No. 11/665,251) (the data presented in Example 2 below demonstrates that administration of ONTAK to rhesus monkeys enhances antibody generation to an antigen).

A further approach to breaking tolerance to administered immunogens is to design the recombinant insert genes with a cytoplasmic domain endoplasmic reticulum retention sequence, such as lysine-lysine, and target the HIV gene (such as Envelope) for retention in the endoplasmic reticulum (Cornall et al, JEM 198:1415-25 (2003)). Such a designed gene can be, for example, a DNA, recombinant adenovirus immunogen or a DNA, recombinant vesicular stomatitis virus immunogen or combinations thereof. Any of a variety of other vectors can also be used to deliver the insert genes (e.g., those presented in Table 1):

TABLE 1
CON-S gp160Cm
MRVRGIQRNCQHLWRWGTLILGMLMICSAAENLWVTVYYGVPVWKEA
NTTLFCASDAKAYDTEVHNVWATHACVPTDPNPQEIVLENVTENFNM
WKNNMVEQMHEDIISLWDQSLKPCVKLTPLCVTLNCTNVNVTNTTNN
TEEKGEIKNCSFNITTEIRDKKQKVYALFYRLDVVPIDDNNNNSSNY
RLINCNTSAITQACPKVSFEPIPIHYCAPAGFAILKCNDKKFNGTGP
CKNVSTVQCTHGIKPVVSTQLLLNGSLAEEEIIIRSENITNNAKTII
VQLNESVEINCTRPNNNTRKSIRIGPGQAFYATGDIIGDIRQAHCNI
SGTKWNKTLQQVAKKLREHFNNKTIIFKPSSGGDLEITTHSFNCRGE
FFYCNTSGLFNSTWIGNGTKNNNNTNDTITLPCRIKQIINMWQGVGQ
AMYAPPIEGKITCKSNITGLLLTRDGGNNNTNETEIFRPGGGDMRDN
WRSELYKYKVVKIEPLGVAPTKAERRVVEREERAVGIGAVFLGFLGA
AGSTMGAASITLTVQARQLLSGIVQQQSNLLRAIEAQQHLLQLTVWG
IKQLQARVLAVERYLKDQQLLGIWGCSGKLICTTTVPWNSSWSNKSQ
DEIWDNMTWMEWEREINNYTDIIYSLIEESQNQQEKNEQELLALDKW
ASLWNWFDITNWLWYIKIFIMIVGGLIGLRIVFAVLSIVNRVRQGYS
PLSFQTLIPNPRGPDRPEGIEEEGGEQDRDRSIRLVNGFLALAWDDL
RSLCLFSYHRLRDFILIAARTVELLGRKGLRRGWEALKYLWNLLQYW
GQELKNSAISLLDTTAIAVAEGTDRVIEVVQRACRAILNIPRRIRQG
LERALL
Fusion domain is in bold and underlined, HR-1
is underlined, HR-2 is in bold, immunodominant
region is in enlarged text and underlined, and
transmembrane domain is in enlarged text. K at
position 494 mutated to E and K at position 502
is mutated to E to delete cleavage site.
CON-S gp160CmKK
MRVRGIQRNCQHLWRWGTLILGMLMICSAAENLWVTVYYGVPVWKEA
NTTLFCASDAKAYDTEVHNVWATHACVPTDPNPQEIVLENVTENFNM
WKNNMVEQMHEDIISLWDQSLKPCVKLTPLCVTLNCTNVNVTNTTNN
TEEKGEIKNCSFNITTEIRDKKQKVYALFYRLDVVPIDDNNNNSSNY
RLINCNTSAITQACPKVSFEPIPIHYCAPAGFAILKCNDKKFNGTGP
CKNVSTVQCTHGIKPVVSTQLLLNGSLAEEEIIIRSENITNNAKTII
VQLNESVEINCTRPNNNTRKSIRIGPGQAFYATGDIIGDIRQAHCNI
SGTKWNKTLQQVAKKLREHFNNKTIIFKPSSGGDLEITTHSFNCRGE
FFYCNTSGLFNSTWIGNGTKNNNNTNDTITLPCRIKQIINMWQGVGQ
AMYAPPIEGKITCKSNITGLLLTRDGGNNNTNETEIFRPGGGDMRDN
WRSELYKYKVVKIEPLGVAPTKAERRVVEREERAVGIGAVFLGFLGA
AGSTMGAASITLTVQARQLLSGIVQQQSNLLRAIEAQQHLLQLTVWG
IKMQARVLAVERYLKDQQLLGIWGCSGKLICTTTVPWNSSWSNKSQD
EIWDNMTWMEWEREINNYTDIIYSLIEESQNQQEKNEQELLALDKWA
SLWNWFDITNWLWYIKIFIMIVGGLIGLRIVFAVLSIVNRVRQGYSP
LSFQTLIPNPRGPDRPEGIEEEGGEQDRDRSIRLVNGFLALAWDDLR
SLCLFSYHRLRDFILIAARTVELLGRKGLRRGWEALKYLWNLLQYWG
QELKNSAISLLDTTAIAVAEGTDRVIEVVQRACRAILNIPRRIRQGL
ERALLKK
Fusion domain in bold and underlined, HR-1 is
underlined, HR-2 is in bold, immunodominant
region is in enlarged text and underlined, and
transmembrane domain is in enlarged text. K at
position 494 mutated to E and K at position 502
is mutated to E to delete cleavage site. KK are
added at the C-terminal end.
CON-S gp160
MRVRGIQRNCQHLWRWGTLILGMLMICSAAENLWVTVYYGVPVWKEA
NTTLFCASDAKAYDTEVHNVWATHACVPTDPNPQEIVLENVTENFNM
WKNNMVEQMHEDIISLWDQSLKPCVKLTPLCVTLNCTNVNVTNTTNN
TEEKGEIKNCSFNITTEIRDKKQKVYALFYRLDVVPIDDNNNNSSNY
RLINCNTSAITQACPKVSFEPIPIHYCAPAGFAILKCNDKKFNGTGP
CKNVSTVQCTHGIKPVVSTQLLLNGSLAEEEIIIRSENITNNAKTII
VQLNESVEINCTRPNNNTRKSIRIGPGQAFYATGDIIGDIRQAHCNI
SGTKWNKTLQQVAKKLREHFNNKTIIFKPSSGGDLEITTHSFNCRGE
FFYCNTSGLFNSTWIGNGTKNNNNTNDTITLPCRIKQIINMWQGVGQ
AMYAPPIEGKITCKSNITGLLLTRDGGNNNTNETEIFRPGGGDMRDN
WRSELYKYKVVKIEPLGVAPTKAKRRVVEREKRAVGIGAVFLGFLGA
AGSTMGAASITLTVQARQLLSGIVQQQSNLLRAIEAQQHLLQLTVWG
IKQLQARVLAVERYLKDQQLLGIWGCSGKLICTTTVPWNSSWSNKSQ
DEIWDNMTWMEWEREINNYTDIIYSLIEESQNQQEKNEQELLALDKW
ASLWNWFDITNWLWYIKIFIMIVGGLIGLRIVFAVLSIVNRVRQGYS
PLSFQTLIPNPRGPDRPEGIEEEGGEQDRDRSIRLVNGFLALAWDDL
RSLCLFSYHRLRDFILIAARTVELLGRKGLRRGWEALKYLWNLLQYW
GQELKNSAISLLDTTAIAVAEGTDRVIEVVQRACRAILNIPRRIRQG
LERALL
Fusion domain is in bold and underlined, HR-1
is underlined, HR-2 is in bold, immunodominant
region is in enlarged text and underlined, and
transmembrane domain is in enlarged text
CON-S gp160KK
MRVRGIQRNCQHLWRWGTLILGMLMICSAAENLWVTVYYGVPVWKEA
NTTLFCASDAKAYDTEVHNVWATHACVPTDPNPQEIVLENVTENFNM
WKNNMVEQMHEDIISLWDQSLKPCVKLTPLCVTLNCTNVNVTNTTNN
TEEKGEIKNCSFNITTEIRDKKQKVYALFYRLDVVPIDDNNNNSSNY
RLINCNTSAITQACPKVSFEPIPIHYCAPAGFAILKCNDKKFNGTGP
CKNVSTVQCTHGIKPVVSTQLLLNGSLAEEEIIIRSENITNNAKTII
VQLNESVEINCTRPNNNTRKSIRIGPGQAFYATGDIIGDIRQAHCNI
SGTKWNKTLQQVAKKLREHFNNKTIIFKPSSGGDLEITTHSFNCRGE
FFYCNTSGLFNSTWIGNGTKNNNNTNDTITLPCRIKQIINMWQGVGQ
AMYAPPIEGKITCKSNITGLLLTRDGGNNNTNETEIFRPGGGDMRDN
WRSELYKYKVVKIEPLGVAPTKAKRRVVEREKRAVGIGAVFLGFLGA
AGSTMGAASITLTVQARQLLSGIVQQQSNLLRAIEAQQHLLQLTVWG
IKQLQARVLAVERYLKDQQLLGIWGCSGKLICTTTVPWNSSWSNKSQ
DEIWDNMTWMEWEREINNYTDIIYSLIEESQNQQEKNEQELLALDKW
ASLWNWFDITNWLWYIKIFIMIVGGLIGLRIVFAVLSIVNRVRQGYS
PLSFQTLIPNPRGPDRPEGIEEEGGEQDRDRSIRLVNGFLALAWDDL
RSLCLFSYHRLRDFILIAARTVELLGRKGLRRGWEALKYLWNLLQYW
GQELKNSAISLLDTTAIAVAEGTDRVIEVVQRACRAILNIPRRIRQG
LERALLKK
JRFL gp160
MRVKGIRKNYQHLWRGGTLLLGIIVICSAVEKLWVTVYYGVPVWKEA
TTTLFCASDAKAYDTEVHNVWATHACVPTDPNPQEVVLGNVTEKFNM
WKNNMVEQMQEDIISLWDQSLKPCVKLTPLCVTLNCKDVNATNTTNG
SEGTMERGEIKNCSFNITTSIRDEVQKEYALFYKLDVVPIDNNNTSY
RLISCDTSVITQACPKISFEPIPIHYCAPAGFAILKCNDKTFNGKGP
CKNVSTVQCTHGIRPVVSTQLLLNGSLAEEEVVIRSDNFTNNAKTII
VQLKESVEINCTRPNNNTRKSIHIGPGRAFYTTGEIIGDIRQAHCNI
SRAKWNDTLKQIVIKLREQFENKTIVFNHSSGGDPEIVMHSFNCGGE
FFYCNSTQLFNSTWNNNTEGSNNTEGNTITLPCRIKQIINMWQEVGK
AMYAPPIRGQIRCSSNITGLLLTRDGGINENGTEIFRPGGGDMKDNW
RSELYKYKVVKIEPLGVAPTKAKRRVVQREKRAVGIGAVFLGFLGAA
GSTMGAASMTLTVQARLLLSGIVQQQNNLLRAIEAQQRMLQLTVWGI
KQLQARVLAVERYLGDQQLLGIWGCSGKLICTTAVPWNASWSNKSLD
RIWNNMTWMEWEREIDNYTSEIYTLIEESQNQQEKNEQELLELDRWA
SLWNWFDITKWLWYIKIFIMIVGGLIGLRIVFTVLSIVNRVRQGYSP
LSFQTLLPAPRGPDRPEGIEEEGGERDRDRSGRLVNGFLALIWVDLR
SLCLFSYHRLRDLLLTVTRIVELLGRRGWEVLKYWWNLLQYWSQELK
NSAVSLLNATAIAVAEGTDRIIEALQRTYRAILHIPTRIRQGLERAL
L
Fusion domain is in bold and underlined, HR-1
is underlined, HR-2 is in bold, immunodominant
region is underlined and contains AVERY
sequence, and transmembrane domain is in
enlarged text.
JRFL gp160KK
MRVKGIRKNYQHLWRGGTLLLGIIVICSAVEKLWVTVYYGVPVWKEA
TTTLFCASDAKAYDTEVHNVWATHACVPTDPNPQEVVLGNVTEKFNM
WKNNMVEQMQEDIISLWDQSLKPCVKLTPLCVTLNCKDVNATNTTNG
SEGTMERGEIKNCSFNITTSIRDEVQKEYALFYKLDVVPIDNNNTSY
RLISCDTSVITQACPKISFEPIPIHYCAPAGFAILKCNDKTFNGKGP
CKNVSTVQCTHGIRPVVSTQLLLNGSLAEEEVVIRSDNFTNNAKTII
VQLKESVEINCTRPNNNTRKSIHIGPGRAFYTTGEIIGDIRQAHCNI
SRAKWNDTLKQIVIKLREQFENKTIVFNHSSGGDPEIVMHSFNCGGE
FFYCNSTQLFNSTWNNNTEGSNNTEGNTITLPCRIKQIINMWQEVGK
AMYAPPIRGQIRCSSNITGLLLTRDGGINENGTEIFRPGGGDMKDNW
RSELYKYKVVKIEPLGVAPTKAKRRVVQREKRAVGIGAVFLGFLGAA
GSTMGAASMTLTVQARLLLSGIVQQQNNLLRAIEAOORMLQLTVWGI
KQLQARVLAVERYLGDQQLLGIWGCSGKLICTTAVPWNASWSNKSLD
RIWNNMTWMEWEREIDNYTSEIYTLIEESQNQQEKNEQELLELDRWA
SLWNWFDITKWLWYIKIFIMIVGGLIGLRIVFTVLSIVNRVRQGYSP
LSFQTLLPAPRGPDRPEGIEEEGGERDRDRSGRLVNGFLALIWVDLR
SLCLFSYHRLRDLLLTVTRIVELLGRRGWEVLKYWWNLLQYWSQELK
NSAVSLLNATAIAVAEGTDRIIEALQRTYRAILHIPTRIRQGLERAL
LKK
Fusion domain is in bold and underlined, HR-1
is underlined, HR-2 is in bold, immunodominant
region is underlined and contains
AVERY sequence, and transmembrane domain
is in enlarged text. KK are added at
the C-terminal end.
JRFL gp160Cm
MRVKGIRKNYQHLWRGGTLLLGIIVICSAVEKLWVTVYYGVPVWKEA
TTTLFCASDAKAYDTEVHNVWATHACVPTDPNPQEVVLGNVTEKFNM
WKNNMVEQMQEDIISLWDQSLKPCVKLTPLCVTLNCKDVNATNTTNG
SEGTMERGEIKNCSFNITTSIRDEVQKEYALFYKLDVVPIDNNNTSY
RLISCDTSVITQACPKISFEPIPIHYCAPAGFAILKCNDKTFNGKGP
CKNVSTVQCTHGIRPVVSTQLLLNGSLAEEEVVIRSDNFTNNAKTII
VQLKESVEINCTRPNNNTRKSIHIGPGRAFYTTGEIIGDIRQAHCNI
SRAKWNDTLKQIVIKLREQFENKTIVFNHSSGGDPEIVMHSFNCGGE
FFYCNSTQLFNSTWNNNTEGSNNTEGNTITLPCRIKQIINMWQEVGK
AMYAPPIRGQIRCSSNITGLLLTRDGGINENGTEIFRPGGGDMKDNW
RSELYKYKVVKIEPLGVAPTKAERRVVQREERAVGIGAVFLGFLGAA
GSTMGAASMTLTVQARLLLSGIVQQQNNLLRAIEAQQRMLQLTVWGI
KQLQARVLAVERYLGDQQLLGIWGCSGKLICTTAVPWNASWSNKSLD
RIWNNMTWMEWEREIDNYTSEIYTLIEESQMQQEKNEQELLELDKWA
SLWNWFDITKWLWYIKIFIMIVGGLIGLRIVFTVLSIVNRVRQGYSP
LSFQTLLPAPRGPDRPEGIEEEGGERDRDRSGRLVNGFLALIWVDLR
SLCLFSYHRLRDLLLTVTRIVELLGRRGWEVLKYWWNLLQYWSQELK
NSAVSLLNATAIAVAEGTDRIIEALQRTYRAILHIPTRIRQGLERAL
L
Fusion domain is in bold and underlined, HR-1
is underlined, HR-2 is in bold, immunodominant
region is in underlined and contains AVERY
sequence, and transmembrane domain is in
enlarged text. K at position 493 mutated to E
and K at position 501 is mutated to EE to
delete cleavage site.
JRFL gp160CmKK
MRVKGIRKNYQHLWRGGTLLLGIIVICSAVEKLWVTVYYGVPVWK
EATTTLFCASDAKAYDTEVHNVWATHACVPTDPNPQEVVLGNVTE
KFNMWKNNMVEQMQEDIISLWDQSLKPCVKLTPLCVTLNCKDVNA
TNTTNGSEGTMERGEIKNCSFNITTSIRDEVQKEYALFYKLDVVP
IDNNNTSYRLISCDTSVITQACPKISFEPIPIHYCAPAGFAILKC
NDKTFNGKGPCKNVSTVQCTHGIRPVVSTQLLLNGSLAEEEVVIR
SDNFTNNAKTIIVQLKESVEINCTRPNNNTRKSIHIGPGRAFYTT
GEIIGDIRQAHCNISRAKWNDTLKQIVIKLREQFENKTIVFNHSS
GGDPEIVMHSFNCGGEFFYCNSTQLFNSTWNNNTEGSNNTEGNTI
TLPCRIKQIINMWQEVGKAMYAPPIRGQIRCSSNITGLLLTRDGG
INENGTEIFRPGGGDMKDNWRSELYKYKVVKIEPLGVAPTKAERR
VVQREERAVGIGAVFLGFLGAAGSTMGAASMTLTVQARLLLSGIV
QQQ[E,UNS NNLLRAIEAQQRMLQLTVWGIKQLQARVLAVERYLGDQ[EE QLLG
IWGCSGKLICTTAVPWNASWSNKSLDRIWNNMTWMEWEREIDNYT
SEIYTLIEESQNQQEKNEQELLELDRWASLWNWFDITKWLWYIKI
FIMIVGGLIGLRIVFTVLSIVNRVRQGYSPLSFQTLLPAPRGPDR
PEGIEEEGGERDRDRSGRLVNGFLALIWVDLRSLCLFSYHRLRDL
LLTVTRIVELLGRRGWEVLKYWWNLLQYWSQELKNSAVSLLNATA
IAVAEGTDRIIEALQRTYRAILHIPTRIRQGLEFTALL
Fusion domain is in bold and underlined, HR-1
is underlined, HR-2 is in bold, immunodominant
region is underlined and contains AVERY
sequence, and transmembrane domain is in
enlarged text. K at position 493 mutated to E
and K at position 501 is mutated to EE to
delete cleavage site. KK are added at the
C-terminal end.
JRFL gp41
MRVRGIQRNCQHLWRWGTLILGMLMICSAARAVGIGAVFLGFLGA
AGSTMGAASMTLTVQARLLLSGIVQQQNNLLRAIEAQORMLOLTV
WGIKQLOARVLAVERYLGDQQLLGIWGCSGKLICTTAVPWNASWS
NKSLDRIWNNMTWMEWEREIDNYTSEIYTLIEESQNQQEKNEQEL
LELDKWASLWNWFDITKWLWYIKIFIMIVGGLVGLRLVFTVLSIV
NRVRQGYSPLSFQTLLPAPRGPDRPEGIEEEGGERDRDRSGRLVN
GFLALIWVDLRSLCLFSYHRLRDLLLTVTRIVELLGRRGWEVLKY
WWNLLQYWSQELKNSAVSLLNATAIAVAEGTDRHEALQRTYRAIL
HIPTRIRQGLERALL
CON-S leader sequence at the N-terminus will
be used as protein synthesis initiation and
maturation signal. Fusion domain is in bold
and underlined, HR-1 is underlined, HR-2 is
in bold, immunodominant region is in enlarged
text and underlined, and transmembrane domain
in enlarged text.
JRFL gp41-KK
MRVRGIQRNCQHLWRWGTLILGMLMICSAARAVGIGAVFLGFLGA
AGSTMGAASMTLTVQARLLLSGIVQQQNNLLRAIEAQQRMLQLTV
WGIKQLQARVLAVERYLGDQQLLGIWGCSGKLICTTAVPWNASWS
NKSLDRIWNNMTWMEWEREIDNYTSEIYTLIEESQNQQEKNEQEL
LELDKWASLWNWFDITKWLWYIKIFIMIVGGLVGLRLVFTVLSIV
NRVRQGYSPLSFQTLLPAPRGPDRPEGIEEEGGERDRDRSGRLVN
GFLALIWVDLRSLCLFSYHRLRDLLLTVTRIVELLGRRGWEVLKY
WWNLLQYWSQELKNSAVSLLNATAIAVAEGTDRIIEALQRTYRAI
LHIPTRIRQGLERALLKK
CON-S leader sequence at the N-terminus will
be used as protein synthesis initiation and
maturation signal. Fusion domain is in bold
and underlined, HR-1 is underlined,
HR-2 is in bold, immunodominant region
is in enlarged text and underlined, and
transmembrane domain is in enlarged text.
KK are added at the C-terminal end.

The diversity of HIV can be addressed by using a consensus (PCT/US2004/030397 and U.S. application Ser. Nos. 10/572,638 and 11/896,934) and/or mosaic (PCT/US2006/032907) gene T cell and B cell vaccine design strategy. Use of these strategies can eliminate much of the inter- and intra-clade diversity of HIV and induce cross clade T and B cell responses to HIV-1 that are superior to wild-type HIV genes (Gaschen et al, Science 296:2354 (2002); Liao et al, Virology 353:268 (2006), Gao et al, J. Virol. 79:1154 (2005), Weaver et al, J. Virol. 80:6754 (2006)). The mosiac gene approach (Fischer et al, Nature Medicine 13(1):100-106 (2007), Epub 2006 Dec. 24; PCT/US2006/032907) uses in silico evolution to design genes that together, when used as an immunogen, provide optimal T cell epitope coverage for inducing anti-HIV T cell responses. Thus, an integral part of the instant HIV vaccine construct is consensus env, gag, pol, nef, and tat genes. Preferred genes include year 2003 group M consensus gene sequences from Los Alamos National Laboratory HIV Sequence Database sequences, or, alternatively, newer consensus gene sequences selected from a transmitted HIV isolate database, such as developed in the Center for HIV AIDS Vaccine Immunology. In addition, use of mosaic HIV genes, such as gag and nef, can be used to broaden the T cell responses to multiple HIV strains. For induction of neutralizing antibodies, Env constructs can be group M consensus year 2001, CON-S, year 2003 CON-T or a newer consensus Env from transmitted HIV strains, for example, in the forms of gp160, gp140C, gp140CF or gp140CFI (Liao et al, Virology 353:268 (2006)) (gp140CFI refers to an HIV-1 envelope design in which the cleavage-site is deleted (C), the fusion-site is deleted (F) and the gp41 immunodominant region is deleted (I), in addition to the deletion of transmembrane and cytoplasmic domains). Alternatively, year 2003 A1 consensus, 2003 Clade C consensus Envs (Tables 2, 3 and 4) can be used for induction of broadly reactive neutralizing antibodies (U.S. application Ser. No. 10/572,638).

TABLE 2
Comparison Of Neutralization Titers Of Guinea Pig Serum Induced
With Subtype A, B Or C Consensus Envs

| | A. con env-03 140CF | | | | CON-B 140CFI | | | | C. con env-03 140CF | | | |
|---|---|---|---|---|---|---|---|---|---|---|---|---|
| HIV-1 Isolate | Guinea Pig Number | | | | Guinea Pig Number | | | | Guinea Pig Number | | | |
| (Subtype) | 1300 | 1301 | 1302 | 1303 | 980 | 1132 | 1098 | 1099 | 1268 | 1269 | 1270 | 1271 |
| B.BX08# | 160 | 176 | 128 | 137 | 66 | <20 | <20 | <20 | <20 | <20 | <20 | <20 |
| B.QH0692.42 | 129 | 157 | 185 | 135 | 44 | 46 | 49 | 53 | 149 | 138 | 76 | 110 |
| B.SS1196.1 | 130 | 449 | 291 | 141 | 1,257 | 717 | 922 | 881 | 402 | >540 | 253 | 484 |
| B.SF162.LS | 8,686 | 20,502 | 12,427 | 9,920 | 11,030 | 6,194 | 5,608 | 15,012 | 37,634 | 41,842 | 16,225 | 16,511 |
| B. BaL.26 | 86 | >540 | 152 | 112 | 364 | 164 | 362 | 304 | 356 | 293 | 134 | 233 |
| B.92US715 | ND | ND | ND | ND | ND | ND | ND | ND | ND | ND | ND | ND |
| B.JRFL-MC** | 24 | 42 | 28 | <20 | 35 | 36 | <20 | <20 | 43 | 45 | <20 | <20 |
| B. 6101 | <20 | <20 | <20 | <20 | <20 | <20 | <20 | <20 | <20 | <20 | 23 | 29 |
| B.7165 | ND | ND | ND | ND | ND | ND | ND | ND | ND | ND | ND | ND |
| QH0515 | ND | ND | ND | ND | ND | ND | ND | ND | ND | ND | ND | ND |
| B.BG1168 | ND | ND | ND | ND | ND | ND | ND | ND | ND | ND | ND | ND |
| B.3968 | <20 | <20 | <20 | <20 | <20 | <20 | <20 | <20 | <20 | <20 | <20 | <20 |
| ARI.29 | ND | ND | ND | ND | ND | ND | ND | ND | ND | ND | ND | ND |
| PAVO | ND | ND | ND | ND | ND | ND | ND | ND | ND | ND | ND | ND |
| TORNO | ND | ND | ND | ND | ND | ND | ND | ND | ND | ND | ND | ND |
| WITO | ND | ND | ND | ND | ND | ND | ND | ND | ND | ND | ND | ND |
| C.TV-1.21 | 720 | 1224 | 1046 | 751 | <20 | <20 | <20 | <20 | 2,029 | 2,547 | 1,939 | 1,699 |
| C.DU123 | 99 | 127 | 86 | 173 | <20 | <20 | <20 | <20 | 107 | 150 | 108 | 136 |
| C.DU172.18 | 241 | 237 | 389 | 251 | <20 | <20 | <20 | <20 | 70 | 121 | 128 | 152 |
| C.DU151 | 69 | 78 | 64 | 62 | <20 | <20 | <20 | <20 | 33 | 50 | 64 | <20 |
| C.DU156 | 97 | 89 | 105 | 69 | <20 | <20 | <20 | <20 | 34 | 85 | 85 | 78 |
| C.DU422 | 63 | 43 | <20 | <20 | <20 | <20 | <20 | <20 | 32 | 51 | 40 | 46 |
| C.97ZA012 | 27 | 31 | 30 | 23 | <20 | <20 | <20 | <20 | <20 | <20 | <20 | <20 |
| C.96ZM651.2 | <20 | 55 | 59 | <20 | 34 | 33 | 36 | 30 | <20 | 149 | <20 | <20 |
| C.92BR025 | 533 | >540 | >540 | >540 | 32 | 42 | 76 | 75 | >540 | >540 | >540 | >540 |
| C.02ZM233M.PB6 | 112 | 119 | 108 | 113 | <20 | <20 | 24 | 21 | 49 | 100 | 87 | 96 |
| C.02ZM197M.PB7 | 78 | 58 | <20 | 66 | <20 | 23 | 29 | 26 | 27 | 64 | 21 | <20 |
| A.92RW020.05 | 57 | 169 | 134 | 137 | 26 | 27 | <20 | <20 | 168 | 229 | 98 | 170 |
| A.92UG037.01 | <20 | 25 | <20 | <20 | 20 | 23 | 23 | 27 | <20 | 26 | 25 | <20 |
| A. Q23 | <20 | <20 | 21 | <20 | <20 | <20 | <20 | <20 | <20 | <20 | <20 | <20 |
| A.Q168 | <20 | 24 | <20 | <20 | <20 | <20 | <20 | <20 | <20 | <20 | <20 | <20 |
| A.Q259 | <20 | <20 | <20 | <20 | <20 | <20 | <20 | <20 | <20 | <20 | <20 | <20 |
| A.Q461 | <20 | 28 | <20 | <20 | <20 | <20 | <20 | <20 | <20 | <20 | <20 | <20 |
| A.Q769 | <20 | <20 | <20 | <20 | <20 | <20 | <20 | <20 | <20 | <20 | <20 | <20 |
| A.Q842 | <20 | <20 | <20 | <20 | <20 | <20 | <20 | <20 | <20 | <20 | <20 | <20 |
| A/E.93TH976 | <20 | 21 | <20 | <20 | <20 | 27 | <20 | <20 | <20 | <20 | <20 | <20 |
| SVA | <20 | <20 | <20 | 107 | <20 | <20 | <20 | <20 | <20 | <20 | <20 | <20 |

50% Neutralization titers were determined in the pseudotype HIV-1 neutralization assay.
Neutralization was considered positive (number in bold) If the titer of post-immune serum was ≧3 fold over the pre-immune bleed serum, and the value of neutralization titer was >30.

TABLE 3
Comparison Of Neutralization Titers Of Guinea Pig Serum Induced With Wild-type A. B Or C Envs

| | 92RWO20 (Subtype A) | | | | JRFL (Subtype B) | | | | 97ZA012 (Subtype C) | | | |
|---|---|---|---|---|---|---|---|---|---|---|---|---|
| HIV-1 Isolate | Guinea Pig Number | | | | Guinea Pig Number | | | | Guinea Pig Number | | | |
| (Subtype) | 854 | 855 | 856 | 857 | 791 | 793 | 796 | 797 | 862 | 863 | 864 | 865 |
| B.BX08# | <20 | <20 | <20 | <20 | 23 | 22 | <20 | <20 | <20 | <20 | <20 | <20 |
| B.QH0692.42 | 34 | <20 | <20 | 36 | 108 | <20 | <20 | <20 | <20 | <20 | <20 | <20 |
| B.SS1196.1 | 115 | 83 | 100 | 150 | 2,203 | 2,095 | 506 | 489 | 23 | 27 | <20 | <20 |
| B.SF162.LS | 1,546 | 412 | 1,301 | 984 | 1,489 | 1,888 | 92 | 290 | 128 | 421 | 88 | 106 |
| B. BaL.26 | ND | ND | ND | ND | ND | ND | ND | ND | ND | ND | ND | ND |
| 92US715 | ND | ND | ND | ND | ND | ND | ND | ND | ND | ND | ND | ND |
| B.JRFL-MC** | <20 | <20 | <20 | <20 | <20 | <20 | <20 | <20 | <20 | <20 | <20 | <20 |
| B.6101 | <20 | <20 | <20 | <20 | <20 | <20 | <20 | <20 | <20 | <20 | <20 | <20 |
| B.7165 | ND | ND | ND | ND | ND | ND | ND | ND | ND | ND | ND | ND |
| QH0515 | ND | ND | ND | ND | ND | ND | ND | ND | ND | ND | ND | ND |
| B.3968 | <20 | <20 | <20 | <20 | <20 | <20 | <20 | <20 | <20 | <20 | <20 | <20 |
| B.BG1168 | <20 | <20 | <20 | <20 | <20 | <20 | <20 | <20 | <20 | <20 | <20 | <20 |
| ARI.29 | ND | ND | ND | ND | ND | ND | ND | ND | ND | ND | ND | ND |
| PAVO | ND | ND | ND | ND | ND | ND | ND | ND | ND | ND | ND | ND |
| TORNO | ND | ND | ND | ND | ND | ND | ND | ND | ND | ND | ND | ND |
| WITO | ND | ND | ND | ND | ND | ND | ND | ND | ND | ND | ND | ND |
| C.TV-1.21 | 540 | 443 | 449 | 711 | <20 | <20 | <20 | <20 | 93 | 148 | <20 | <20 |
| C.DU123 | 41 | <20 | 48 | 37 | <20 | <20 | <20 | <20 | <20 | 115 | <20 | <20 |
| C.DU172.17 | <20 | <20 | <20 | <20 | <20 | <20 | <20 | <20 | <20 | <20 | <20 | <20 |
| C.DU1512 | <20 | <20 | <20 | <20 | <20 | <20 | <20 | <20 | <20 | <20 | <20 | <20 |
| C.DU156.12 | <20 | <20 | <20 | <20 | <20 | <20 | <20 | <20 | <20 | <20 | <20 | <20 |
| C.DU422.01 | <20 | <20 | <20 | <20 | <20 | <20 | <20 | <20 | <20 | <20 | <20 | <20 |
| C.97ZA012.29 | <20 | <20 | <20 | <20 | <20 | <20 | 36 | 20 | <20 | <20 | <20 | <20 |
| C.96ZM651.2 | <20 | <20 | <20 | <20 | <20 | <20 | <20 | <20 | <20 | <20 | <20 | <20 |
| C.92BR025.9 | 403 | 168 | 258 | 311 | <20 | <20 | <20 | <20 | 55 | 50 | <20 | 39 |
| C.02ZM233M.PB6 | <20 | <20 | <20 | <20 | <20 | <20 | <20 | <20 | <20 | <20 | <20 | <20 |
| C.02ZM197M.PB7 | <20 | <20 | <20 | 27 | 23 | 22 | <20 | <20 | 21 | 22 | <20 | <20 |
| A.92RW020.05 | 150 | 71 | 100 | 106 | <20 | <20 | <20 | <20 | <20 | <20 | <20 | <20 |
| A.92UG037.01 | <20 | <20 | <20 | <20 | <20 | <20 | <20 | <20 | <20 | <20 | <20 | <20 |
| A.Q23 | <20 | <20 | <20 | <20 | <20 | <20 | <20 | <20 | <20 | <20 | <20 | <20 |
| A.Q168 | <20 | <20 | <20 | <20 | <20 | <20 | <20 | <20 | <20 | <20 | <20 | <20 |
| A.Q259 | <20 | <20 | <20 | <20 | <20 | <20 | <20 | <20 | <20 | <20 | <20 | <20 |
| A.Q461 | <20 | <20 | <20 | <20 | <20 | <20 | <20 | <20 | <20 | <20 | <20 | <20 |
| A.Q769 | <20 | <20 | <20 | <20 | <20 | <20 | <20 | <20 | <20 | <20 | <20 | <20 |
| A.Q842 | <20 | <20 | <20 | <20 | <20 | <20 | <20 | <20 | <20 | <20 | <20 | <20 |
| A/E.93TH976 | <20 | <20 | <20 | <20 | <20 | <20 | <20 | <20 | <20 | <20 | <20 | <20 |
| SVA | ND | ND | ND | ND | ND | ND | ND | ND | ND | ND | ND | ND |

50% Neutralization titers were determined in the pseudotype HIV-1 neutralization assay.
Neutralization was considered positive (number in bold) If the titer of post-immune serum was ≧3 fold over the pre-immune bleed serum, and the value of neutralization titer was >30.

TABLE 4
Comparison Of Neutralization Titers Of Guinea Pig Serum Induced With CON-T and CON-S Env

| | CON-T gp140CF/oCpG | | | | CON-S gp140CFI/oCpG | | | |
|---|---|---|---|---|---|---|---|---|
| HIV-1 Isolate | Guinea Pig Number | | | | Guinea Pig Number | | | |
| (Subtype) | 1156 | 1162 | 1163 | 1164 | 963 | 964 | 965 | 966 |
| B.BX08# | <20 | <20 | <20 | <20 | 160 | 211 | 213 | 260 |
| B.QH0692.42 | 54 | 42 | 31 | 43 | 79 | 165 | 111 | 149 |
| B.SS1196.1 | 105 | 136 | 99 | 125 | 916 | 2,760 | 1,471 | 2,822 |
| B.SF162.LS | 7,426 | 7,079 | 5,166 | 3,917 | 43,740 | 43,740 | 43,740 | 43,740 |
| B. BaL.26 | 46 | 93 | 74 | 60 | 354 | 1,021 | 2,056 | 1,161 |
| 92US715 | ND | ND | ND | ND | 37 | 60 | 116 | 40 |
| B.JRFL-MC** | <20 | <20 | <20 | <20 | <20 | <20 | 40 | <20 |
| B.6101 | <20 | <20 | <20 | <20 | <20 | <20 | <20 | <20 |
| B.7165 | ND | ND | ND | ND | 29 | 35 | 63 | 37 |
| QH0515 | ND | ND | ND | ND | <20 | <20 | <20 | <20 |
| B.3968 | <20 | <20 | <20 | <20 | 47 | 59 | 90 | 42 |
| B.BG1168 | ND | ND | ND | ND | <20 | <20 | <20 | <20 |
| ARI.29 | ND | ND | ND | ND | 25 | 36 | 42 | <20 |
| PAVO | ND | ND | ND | ND | 32 | 49 | 75 | <20 |
| TORNO | ND | ND | ND | ND | <20 | 41 | 50 | <20 |
| WITO | ND | ND | ND | ND | 68 | 99 | 100 | 52 |
| C.TV-1.21 | 988 | 430 | 438 | 611 | 1,299 | 2,899 | 1,659 | 4,195 |
| C.DU123 | 153 | >540 | 54 | 46 | 152 | 315 | 127 | >540 |
| C.DU172.17 | 269 | 121 | 126 | <20 | 150 | 130 | 141 | 169 |
| C.DU151.2 | 79 | 52 | 46 | 58 | 53 | 45 | 66 | 48 |
| C.DU156.12 | <20 | 23 | 29 | 90 | 35 | 59 | <20 | 61 |
| C.DU422.01 | <20 | <20 | <20 | <20 | 55 | 57 | 81 | 42 |
| C.97ZA012.29 | <20 | <20 | <20 | <20 | <20 | <20 | <20 | <20 |
| C.96ZM651.2 | 62 | 80 | 45 | 60 | 230 | 261 | 156 | 229 |
| C.928R025.9 | >540 | 377 | 384 | 496 | 3,503 | 6,297 | 3,916 | 5,542 |
| C.02ZM233M.PB6 | <20 | <20 | <20 | <20 | 80 | 150 | 89 | 108 |
| C.02ZM197M.PB7 | <20 | <20 | <20 | <20 | <20 | <20 | <20 | <20 |
| A.92RW020.05 | <20 | <20 | <20 | <20 | 129 | 306 | 180 | 285 |
| A.92UG037.01 | <20 | <20 | <20 | <20 | <20 | <20 | <20 | <20 |
| A.Q23 | <20 | <20 | <20 | <20 | <20 | <20 | <20 | <20 |
| A.Q168 | <20 | <20 | <20 | <20 | <20 | <20 | <20 | <20 |
| A.Q259 | <20 | <20 | <20 | <20 | <20 | <20 | <20 | <20 |
| A.Q461 | <20 | <20 | <20 | <20 | <20 | <20 | <20 | <20 |
| A.Q769 | <20 | <20 | <20 | <20 | <20 | <20 | <20 | <20 |
| A.Q842 | <20 | <20 | <20 | <20 | <20 | <20 | <20 | <20 |
| A/E.93TH976 | <20 | <20 | <20 | <20 | <20 | <20 | <20 | <20 |
| SVA | <20 | <20 | <20 | <20 | ND | ND | ND | ND |
| | CON-S gp140CFI/RIBI | | | | CON-S gp140CFIRIBI | | | |
| HIV-1 Isolate | Guinea Pig Number | | | | Guinea Pig Number | | | |
| (Subtype) | 776 | 777 | 778 | 780 | 871 | 872 | 873 | 874 |
| B.BX08# | 1,196 | 412 | 4,856 | 1,817 | 10 | 116 | 233 | 118 |
| B.QH0692.42 | 109 | <20 | <20 | <20 | 41 | 33 | 74 | 54 |
| B.SS1196.1 | 796 | 296 | 1,339 | 423 | 2,224 | 170 | 415 | 986 |
| B.SF162.LS | 31,224 | 8,186 | 41,667 | 13,369 | 23,619 | 9,916 | 22,467 | 18,639 |
| B. BaL.26 | 444 | 159 | 916 | 444 | 2,195 | 463 | 1,456 | 1,219 |
| 92US715 | 40 | 40 | 33 | <20 | 58 | 42 | 50 | 55 |
| B.JRFL-MC** | <20 | <20 | <20 | <20 | <20 | <20 | <20 | <20 |
| B.6101 | <20 | <20 | <20 | <20 | <20 | <20 | <20 | <20 |
| B.7165 | 90 | 113 | 67 | 44 | 91 | 69 | 113 | 146 |
| QH0515 | <20 | <20 | <20 | <20 | <20 | <20 | <20 | <20 |
| B.3968 | <20 | <20 | <20 | <20 | <20 | <20 | <20 | <20 |
| B.BG1168 | <20 | <20 | <20 | <20 | <20 | <20 | <20 | <20 |
| ARI.29 | ND | ND | ND | ND | 51 | 52 | 74 | 81 |
| PAVO | ND | ND | ND | ND | 32 | 49 | 75 | <20 |
| TORNO | ND | ND | ND | ND | <20 | 41 | 50 | <20 |
| WITO | ND | ND | ND | ND | 68 | 99 | 100 | 52 |
| C.TV-1.21 | 1,339 | 770 | 2,442 | 724 | 2,195 | 463 | 1,486 | 1,219 |
| C.DU123 | 176 | 329 | 387 | 378 | 46 | 49 | 104 | 51 |
| C.DU172.17 | <20 | 235 | <20 | 213 | <20 | <20 | <20 | <20 |
| C.DU151.2 | <20 | <20 | <20 | <20 | 33 | <20 | <20 | <20 |
| C.DU156.12 | <20 | <20 | <20 | <20 | <20 | <20 | <20 | <20 |
| C.DU422.01 | <20 | <20 | <20 | <20 | <20 | <20 | <20 | <20 |
| C.97ZA012.29 | <20 | <20 | <20 | <20 | 27 | 30 | 24 | 31 |
| C.96ZM651.2 | <20 | 22 | <20 | <20 | <20 | <20 | <20 | <20 |
| C.928R025.9 | 1,819 | 1,408 | 3,207 | 1,336 | 2,003 | 540 | 1,724 | 1,598 |
| C.02ZM233M.PB6 | 84 | 61 | 86 | 43 | <20 | <20 | <20 | <20 |
| C.02ZM197M.PB7 | <20 | 33 | 30 | <20 | <20 | 58 | 137 | <20 |
| A.92RW020.05 | 116 | 204 | 95 | 177 | <20 | <20 | 111 | 70 |
| A.92UG037.01 | <20 | <20 | <20 | <20 | <20 | <20 | <20 | <20 |
| A.Q23 | <20 | <20 | <20 | <20 | <20 | <20 | 22 | 22 |
| A.Q168 | <20 | <20 | <20 | <20 | 23 | 20 | 25 | 31 |
| A.Q259 | <20 | <20 | <20 | <20 | <20 | <20 | 22 | 22 |
| A.Q461 | <20 | <20 | <20 | <20 | <20 | <20 | <20 | <20 |
| A.Q769 | <20 | <20 | <20 | <20 | 21 | 21 | 31 | <20 |
| A.Q842 | <20 | <20 | <20 | <20 | <20 | <20 | <20 | <20 |
| A/E.93TH976 | <20 | <20 | <20 | <20 | <20 | <20 | <20 | <20 |
| SVA | ND | ND | ND | ND | ND | ND | ND | ND |

Vectors to be used to administer the HIV-1 genes include DNA for priming (Letvin et al. Science 312:1530-33 (2006)), recombinant adenovirus for boosting (Barouch et al, Nature 441:239-43 (2006), Letvin et al, Science 312:1530-33 (2006), Thorner et al, J. Virol. Epub. Oct. 11, 2006), recombinant vesicular stomatitis virus (Publicover et al, J. Virol. 79:13231-8 (2005)) and recombinant mycobacteria such as attenuated TB, rBCG or rM. smegmatis (Hovav et al, J. Virol., epub., Oct. 18, 2006, Yu et al, Clin. Vacc. Immunol. 13:1204-11 (2006); Derrick et al, Immunology, epub. Oct. 31, 2006). Any of these vectors can be used in prime/boost combinations, and the route of immunization can be systemic (e.g., IM. SC) or mucosal (po. IN, Intravaginally, Intrarectally).

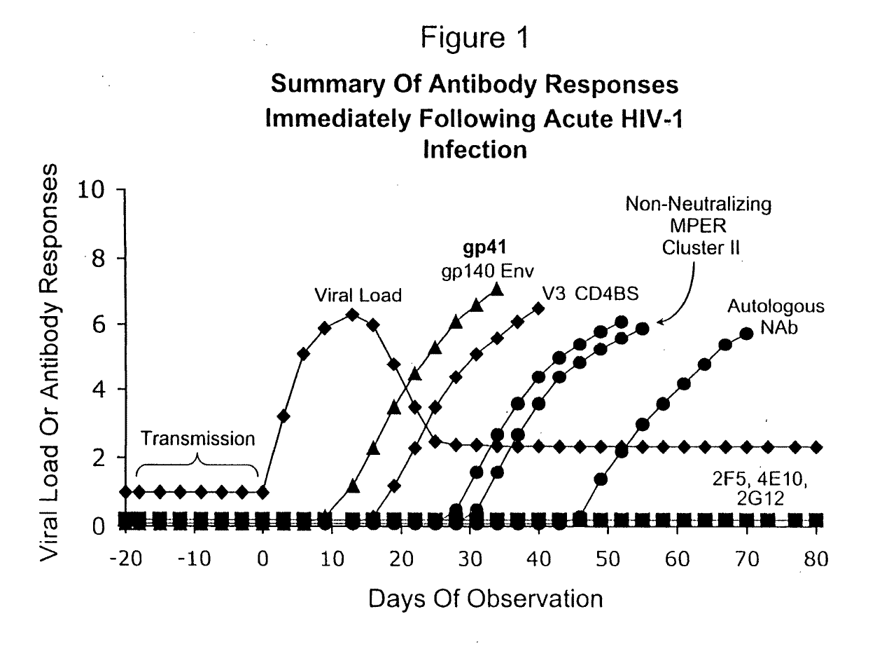

As pointed out above, the present vaccination approach includes a component for overcoming HIV-1 induced apoptosis and immunosuppression to eliminate the delay in T and B cell responses following HIV-1 transmission at mucosal sites. It has recently been shown that while multiple antibody species arise very early in acute HIV infection, non-neutralizing anti-gp41 antibodies arise the earliest, and autologous neutralizing antibodies do not arise until months after transmission (FIG. 1) (Wei, Nature 422:307-12 (2003), Richman Proc. Natl. Acad. Sci. USA 100:4144-9 (2003)). Given the massive apoptosis that occurs coincident with infection and plasma viral load ramp-up in rhesus monkeys infected with SIV, the question has been raised as to whether such a massive apoptosis of immune cells occurs at the earliest stages of human acute HIV infection: Apoptosis is mediated most commonly by members of the tumor necrosis receptor family, including Fas (CD95) and Fas Ligand (CD178), TNF receptors I and II, and TNF-related apoptosis inducing ligand (TRAIL).

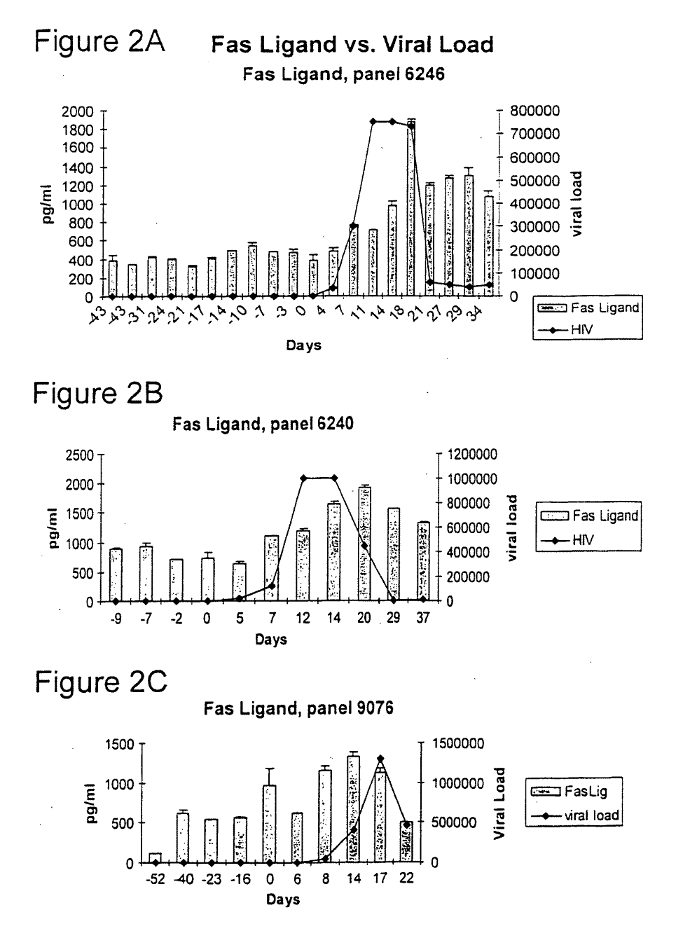

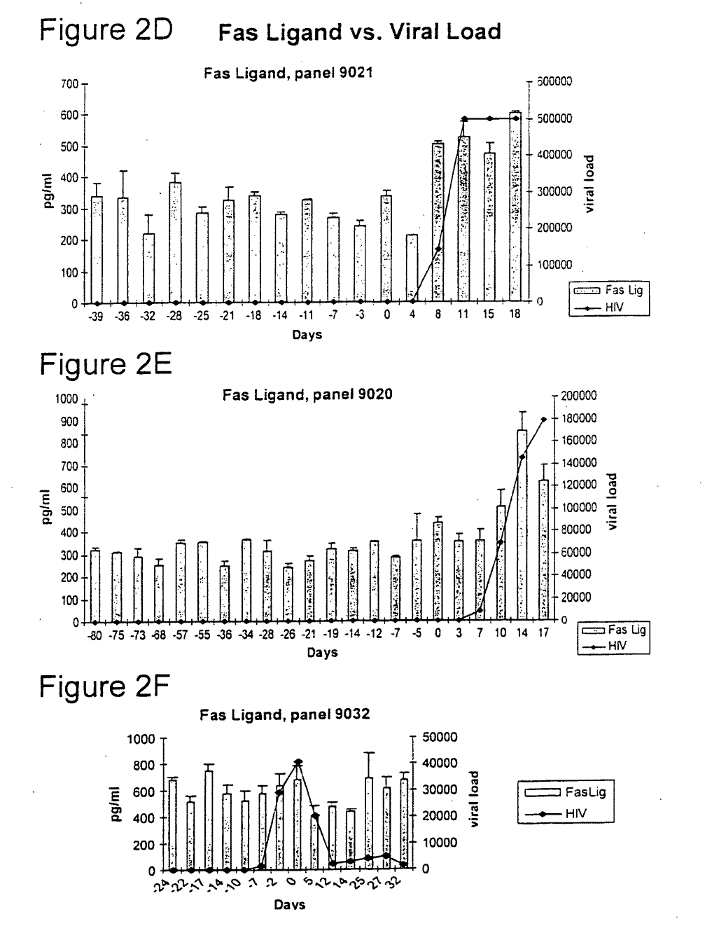

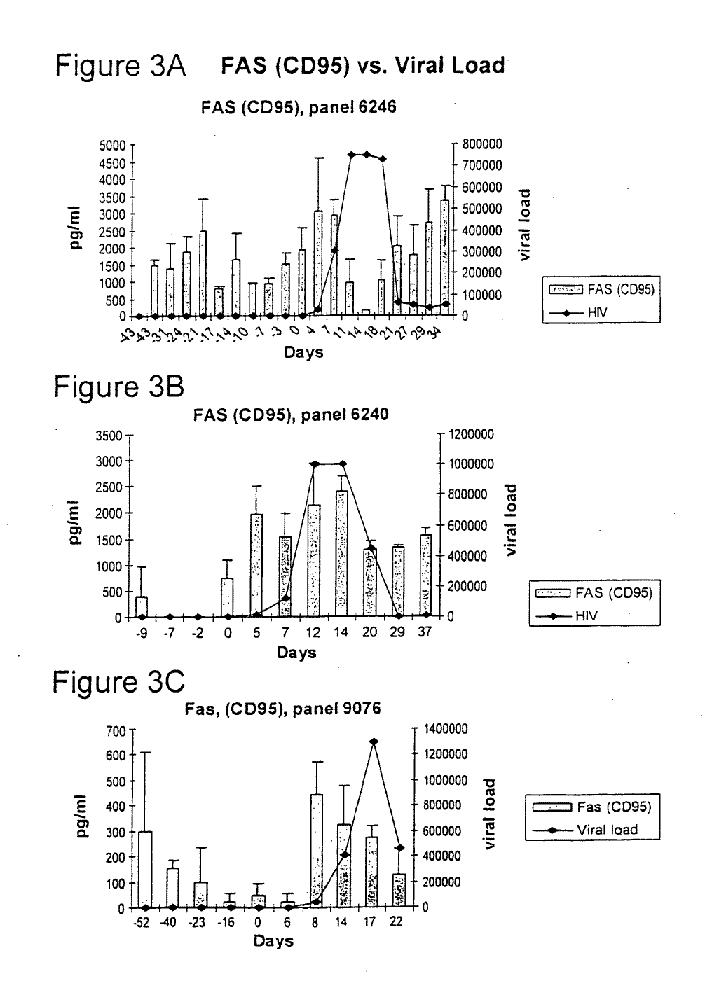

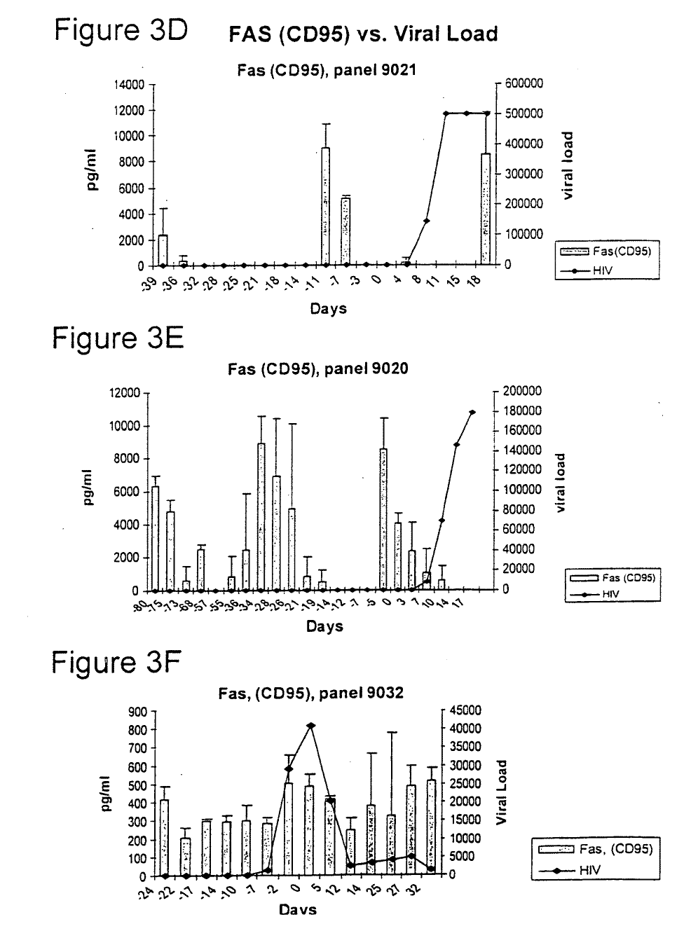

Fas and FasL are dysregulated in chronic HIV-1 infection (Cossarizza et al, AIDS14:346 (2000); Westendorp et al, Nature 375:497 (1995); Sloand et al, Blood 89:1357 (1997)). Studies have been undertaken to determine if there are elevations in plasma Fas or FasL in acute HIV infection. It has been found that, in many AHI patients, there is a dramatic rise in plasma FasL coincident with the rise in plasma viral load (FIG. 2). In addition, in several, but not all, patients there are concomitant rises in plasma Fas (FIG. 3).

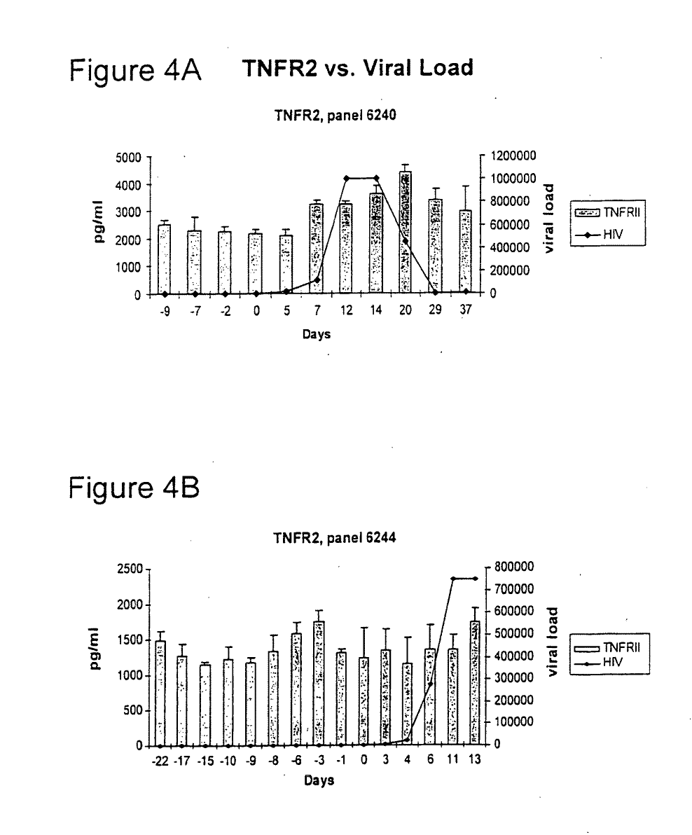

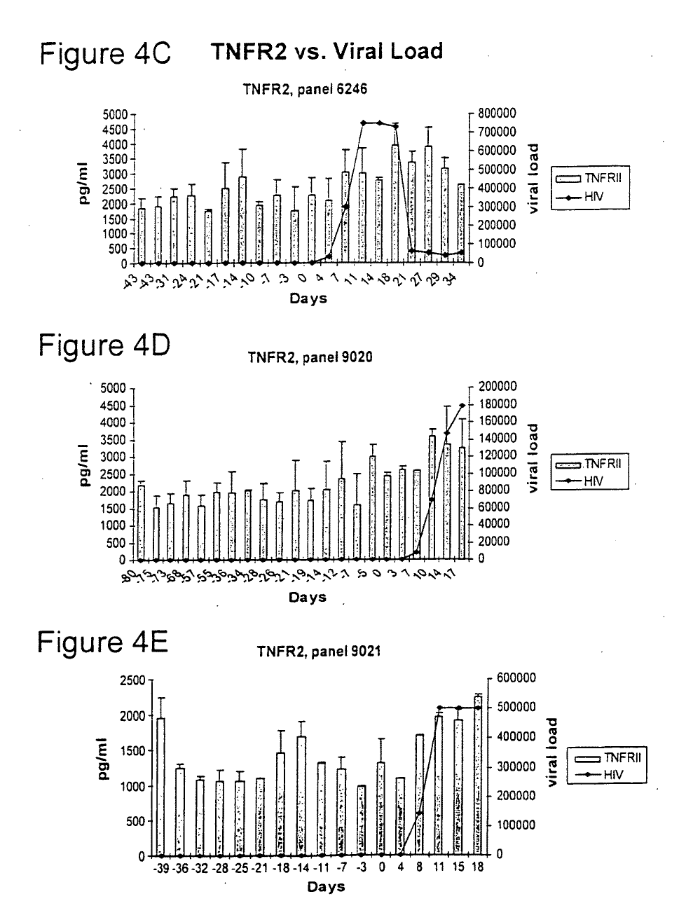

TNFR2 levels are increased in chronic HIV and are predictive of disease progression (Zangerle et al. Immunol Lett. 41:229 (1994)) and TNFR2 is triggered at an early stage of interaction of HIV with monocytes (Rimaniol et al, Cytokine 9:9-18 (1997)). As shown in FIG. 4, soluble TNFR2 elevations have been found in a number of AHI patients during the infection process coincident with the ramp-up of viral load.

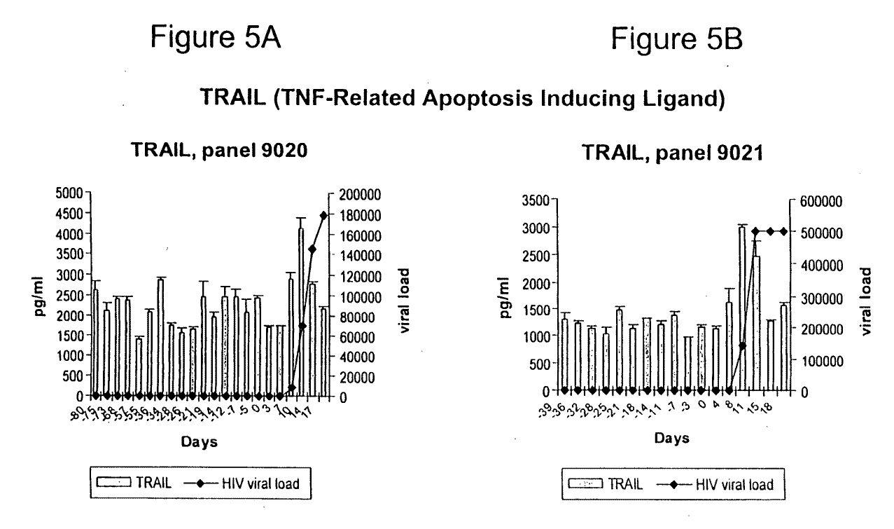

Finally, TRAIL mediates apoptosis of uninfected T cells during HIV infection (Kasich et al, JEM186:1365 (1997); Miura et al, J. Exp. Med. 193: 51 (2001)). FIG. 5 shows that plasma TRAIL levels are elevated in AHI as well.

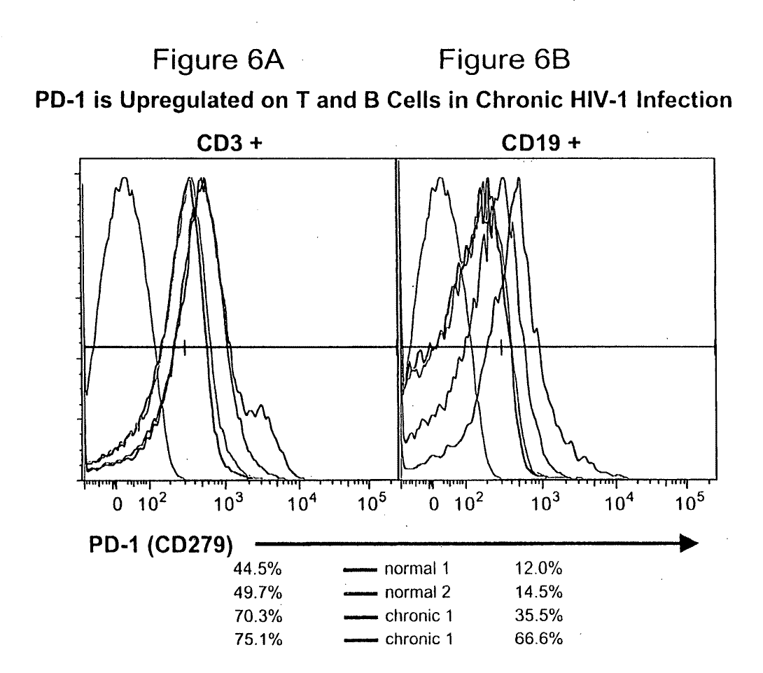

Thus, HIV virions and HIV envelope can directly induce T cell death in AHI, soluble TRAIL can bind to uninfected cells and induce death in AHI, and with both HIV infection of cells and with massive apoptosis, high levels of phosphatidylserine containing cells and particles likely abound in AHI. It has recently been shown that PD-1 (programmed death molecule-1) is present on the surface of human B cells in chronic HIV infection. This suggests that human B cells are primed for apoptosis in HIV infection (FIG. 6). HIV specific CD8+T cell PD-1 expression correlates with a CD8+T cell response to poorly controlled chronic HIV infection (Petrovas et al, TEM 203: 2281 (2006)).

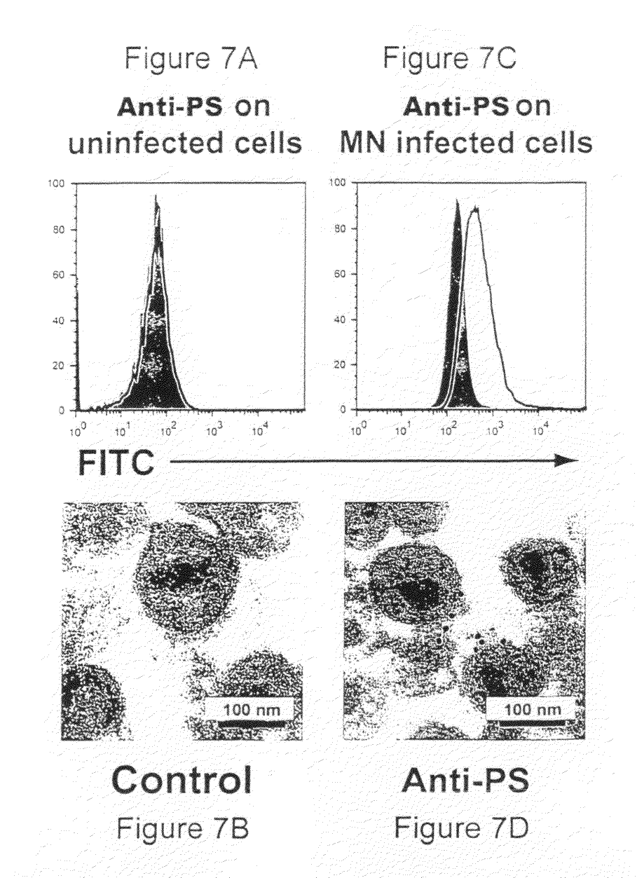

Phosphatidylserine (PS) on the surface of HIV infected cells and virions has been found (FIG. 7) and Callahan et al have found PS is a cofactor for HIV infection of monocytes (Callahan, J. Immunol 170:4840 (2003)). PS-dependent ingestion of apoptotic cells promotes TGF-β1 secretion (Huynh et al, J. Clin. Invest. 109:41 (2002)) and interaction between PS and PS receptor inhibits antibody responses in vivo (Hoffman et al, J. Immunol. 174:1393 (2005)). INF-α, an anti-viral cytokine, sensitizes lymphocytes to apoptosis (Carrero et al. JEM 200:535 (2004)). There are increases in PS+ shed membrane particles in chronic HIV infection (Aupeix et al, J. Clin. Invest. 99:1546 (1997)), and apoptotic microparticles modulate macrophage immune responses (Distler et al, Apoptosis 10:731 (2005)). Apoptotic microparticles are profoundly proinflammatory (Distler et al, PNAS 102:2892 (2005)) and induction of proinflammatory cytokines fuels the HIV infection and virion production process. Oxidized PS-CD36 interactions play an essential role in macrophage dependent phagocytosis of apoptotic cells, and B cells also express CD36 (Greenberg et al, JEM, Nov. 13, 2006, online pub).

Thus, the massive apoptosis that occurs with acute HIV infection with resulting release of TRAIL, mediation of apoptosis via FAS-FASL interactions, and release of PS containing viral and other particles all conspire to initially immunosuppress the host, preventing rapid protective B cell responses.

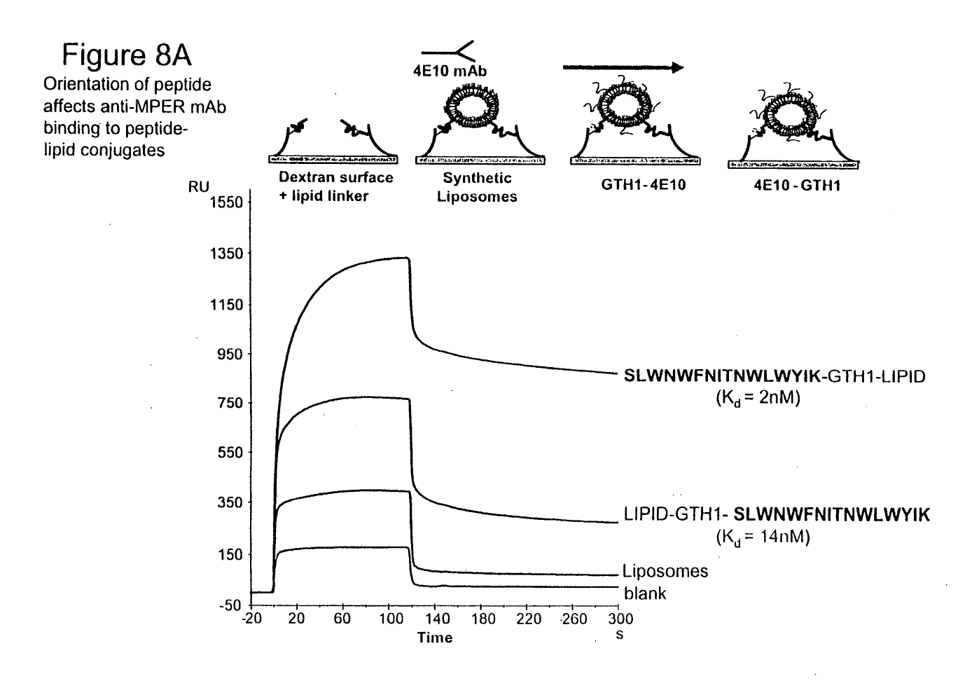

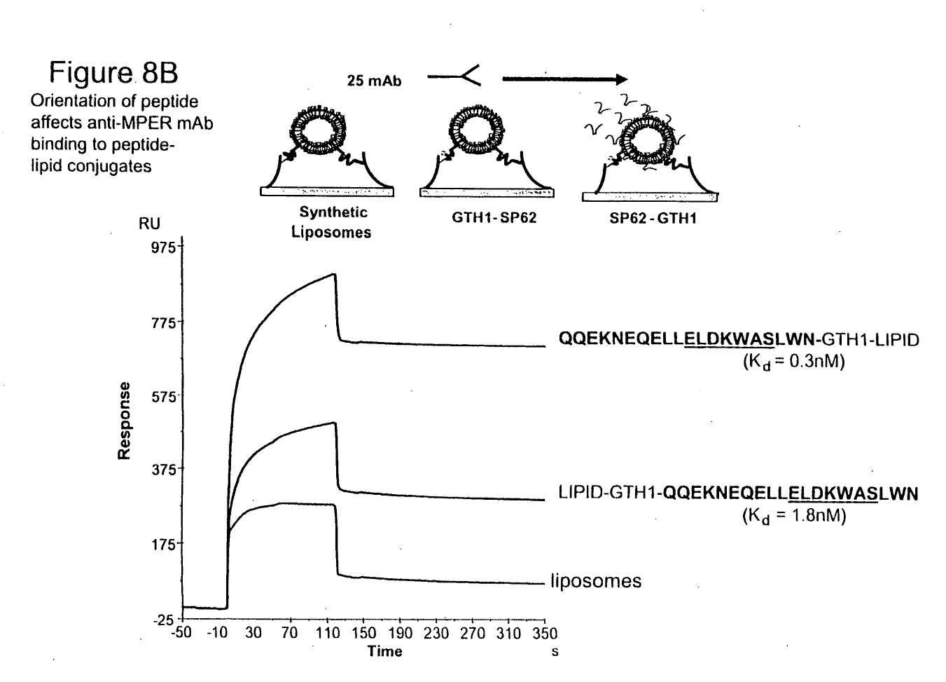

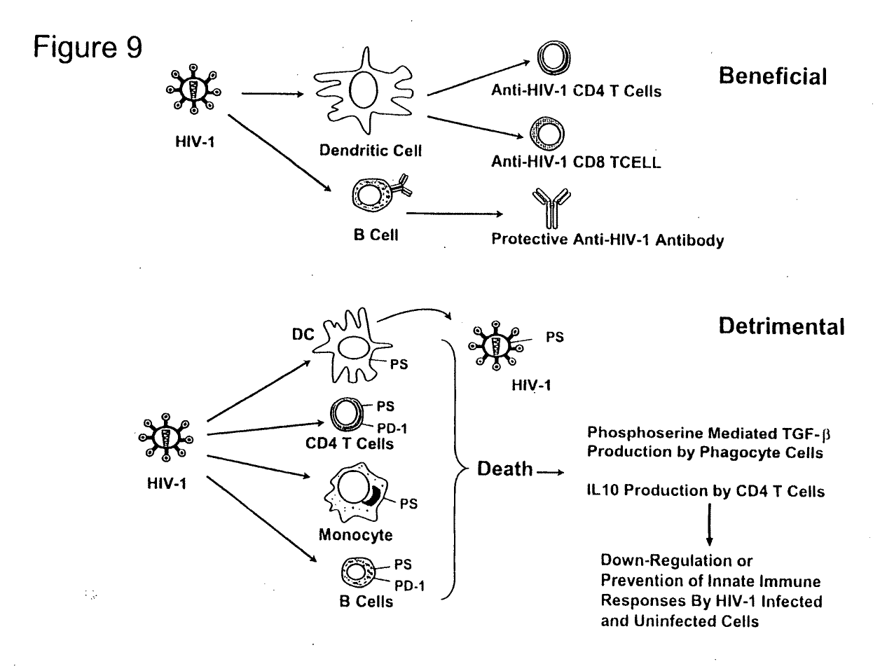

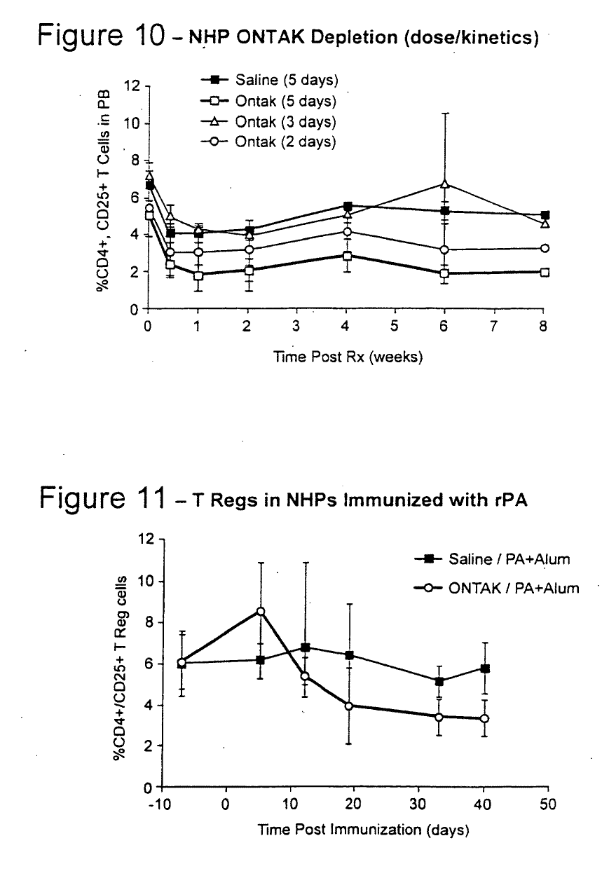

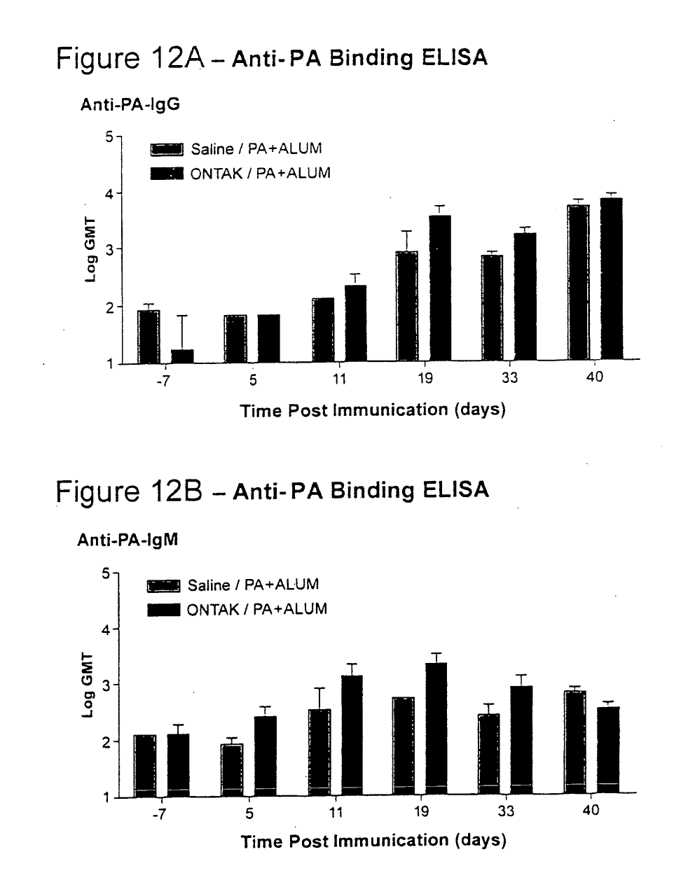

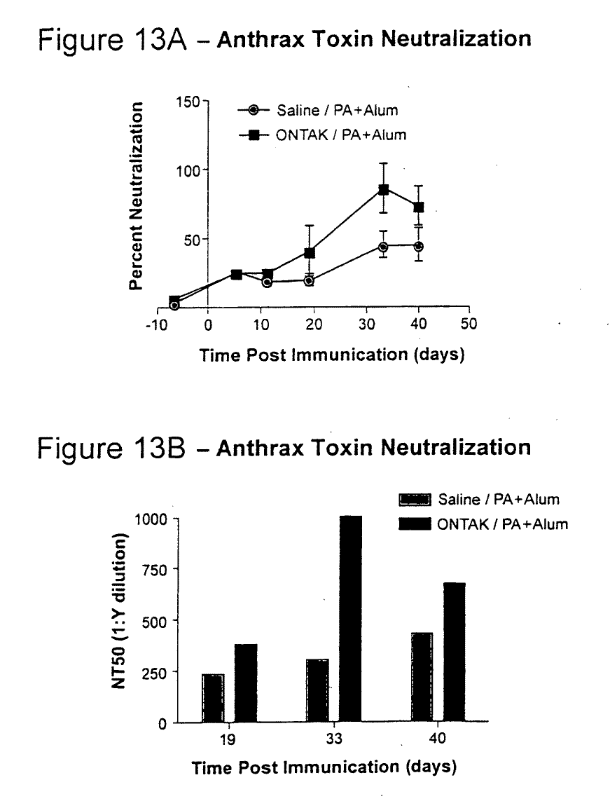

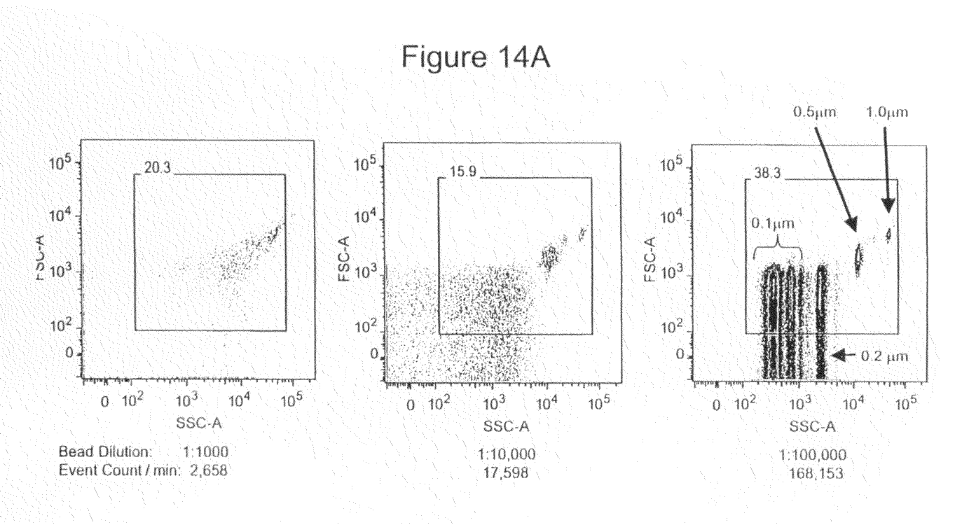

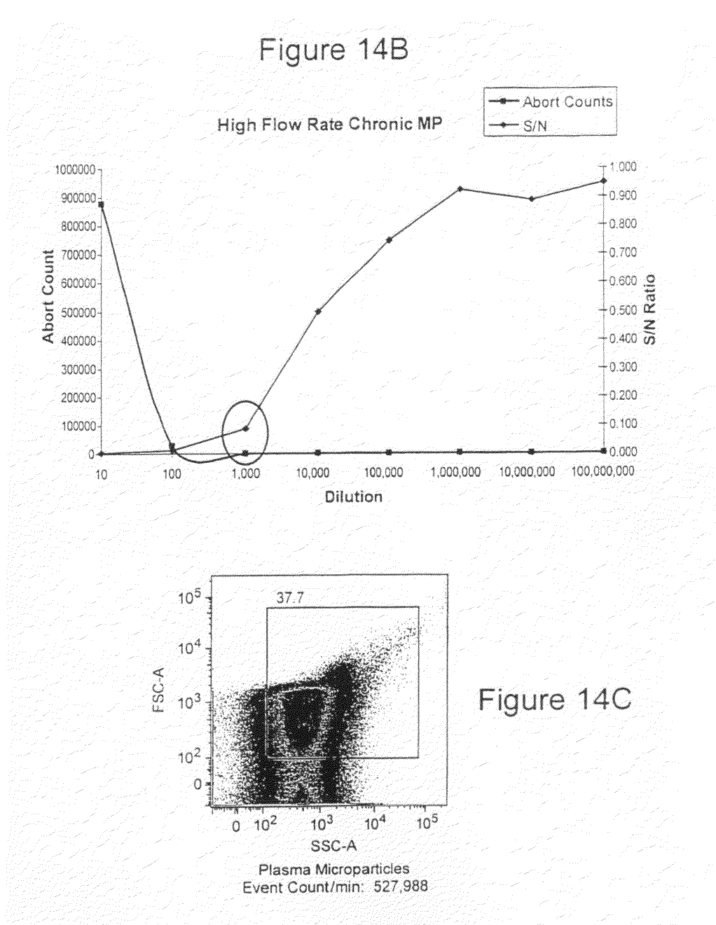

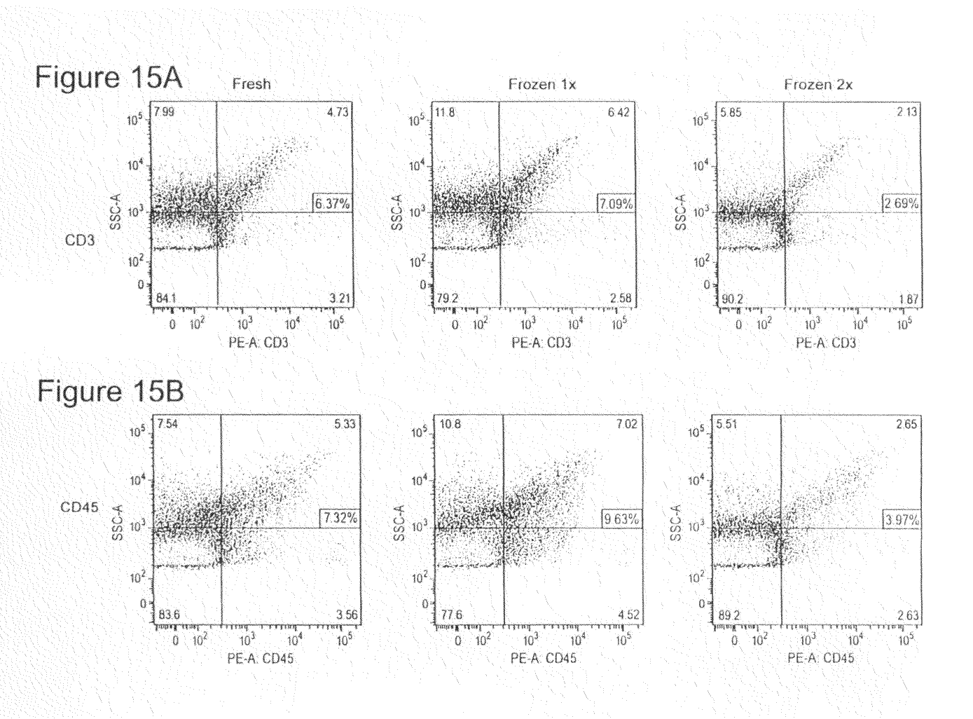

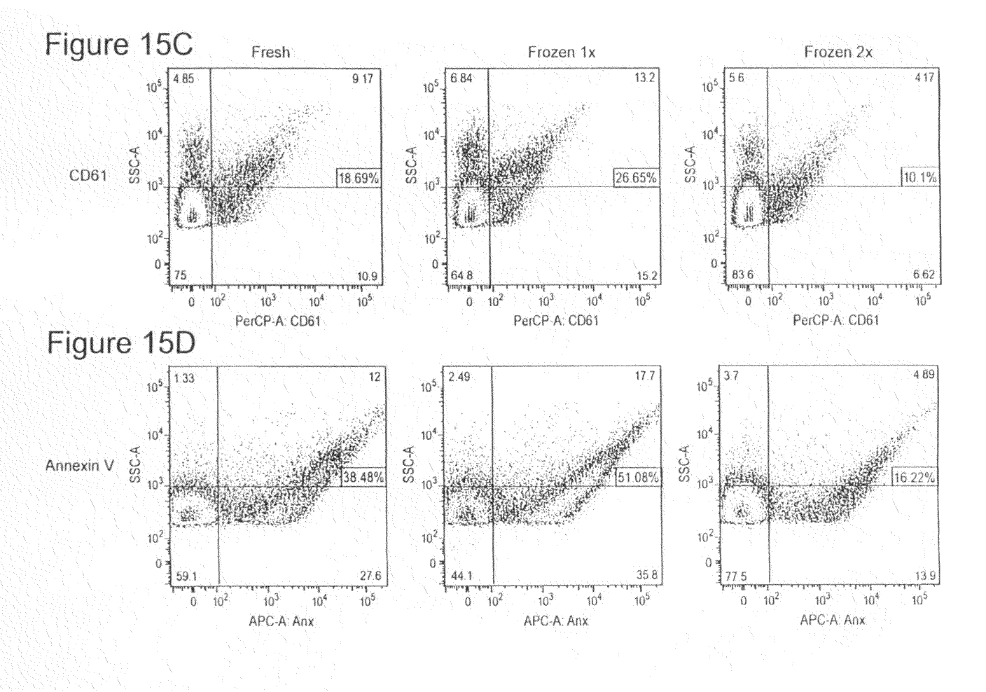

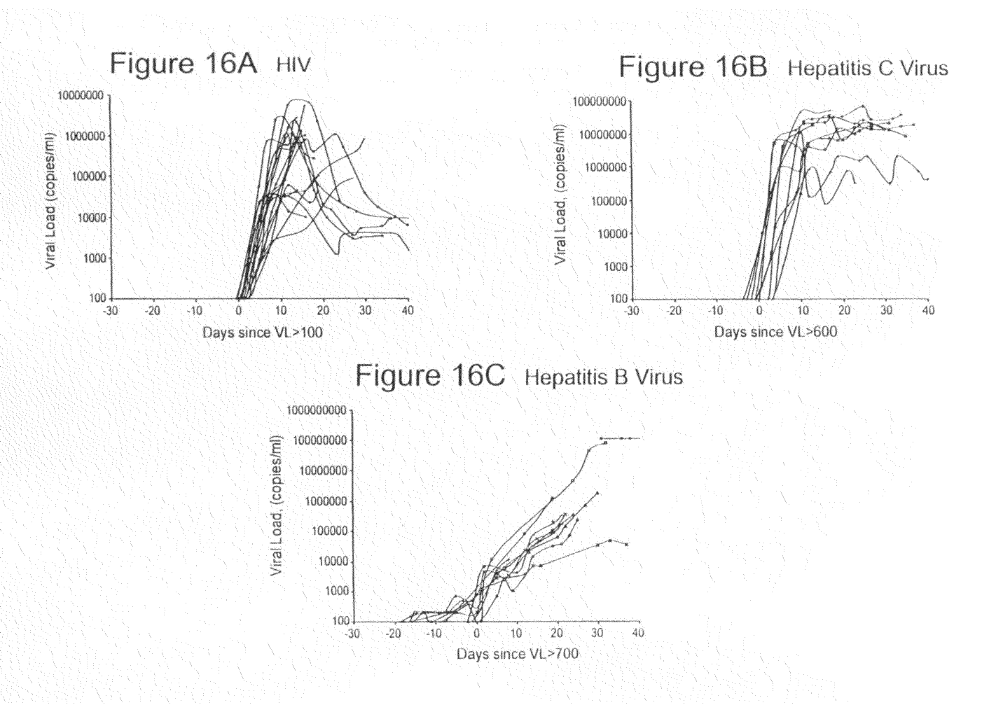

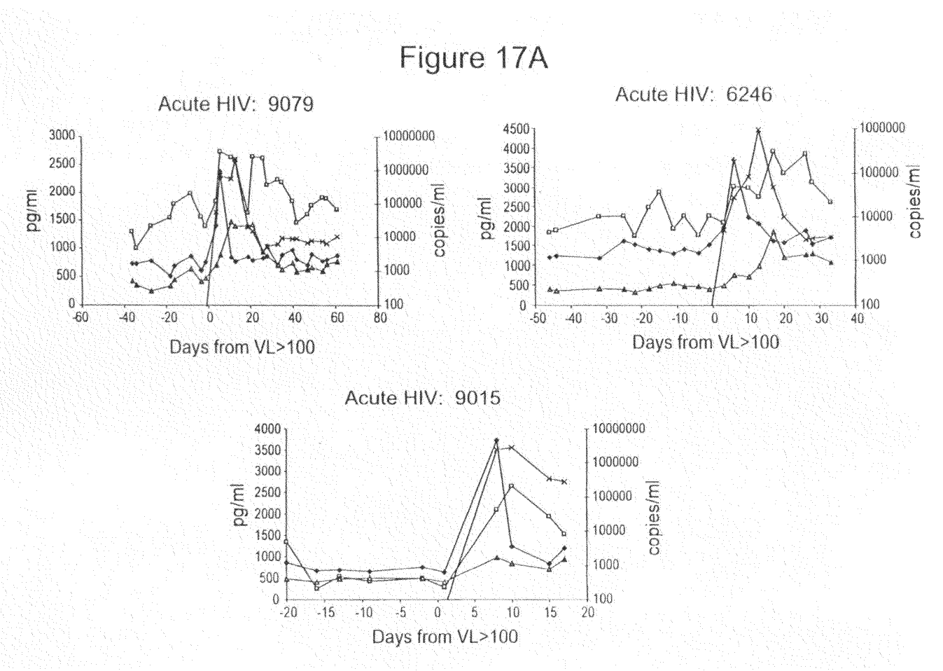

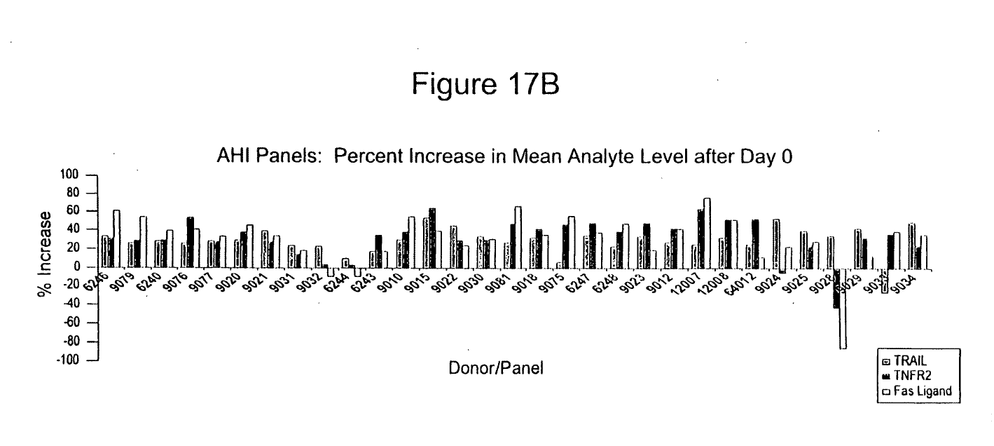

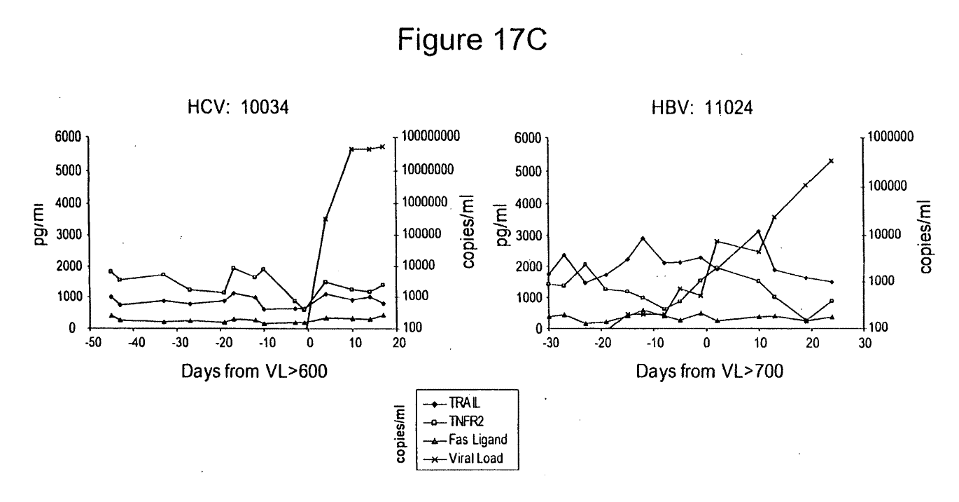

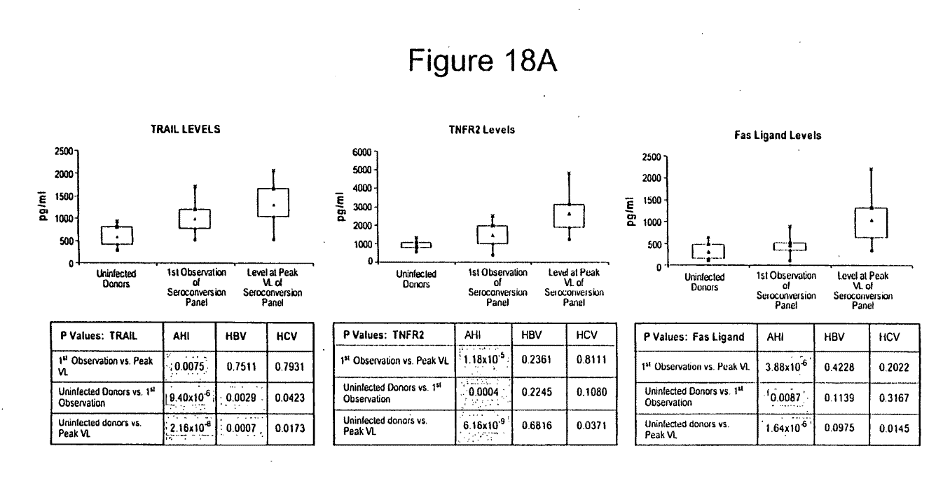

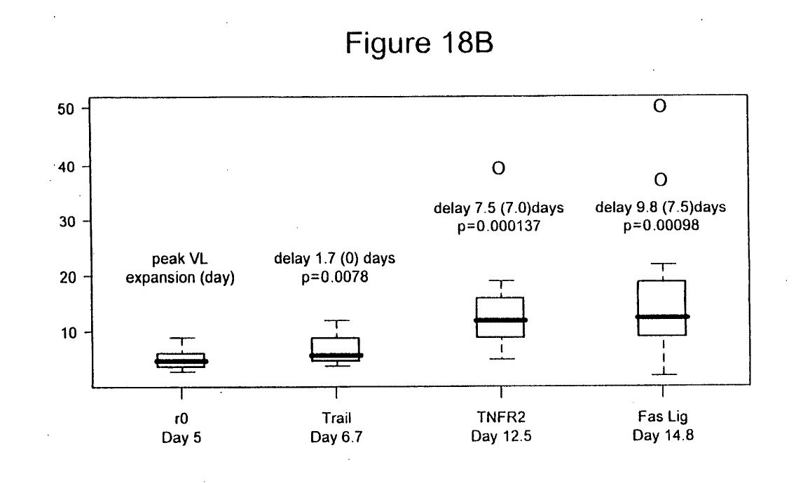

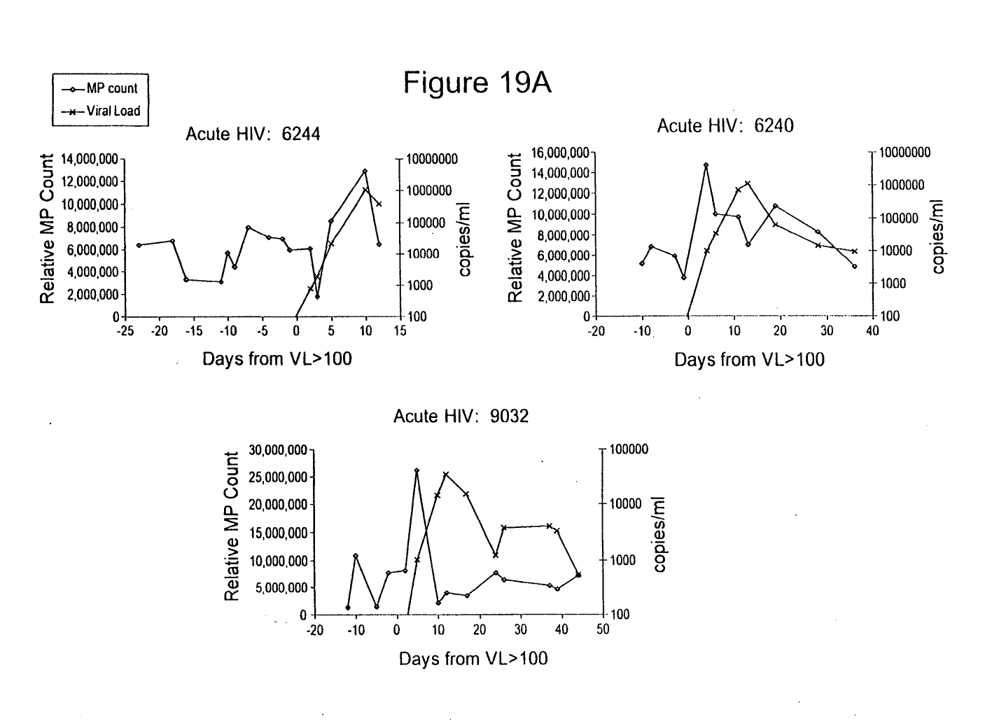

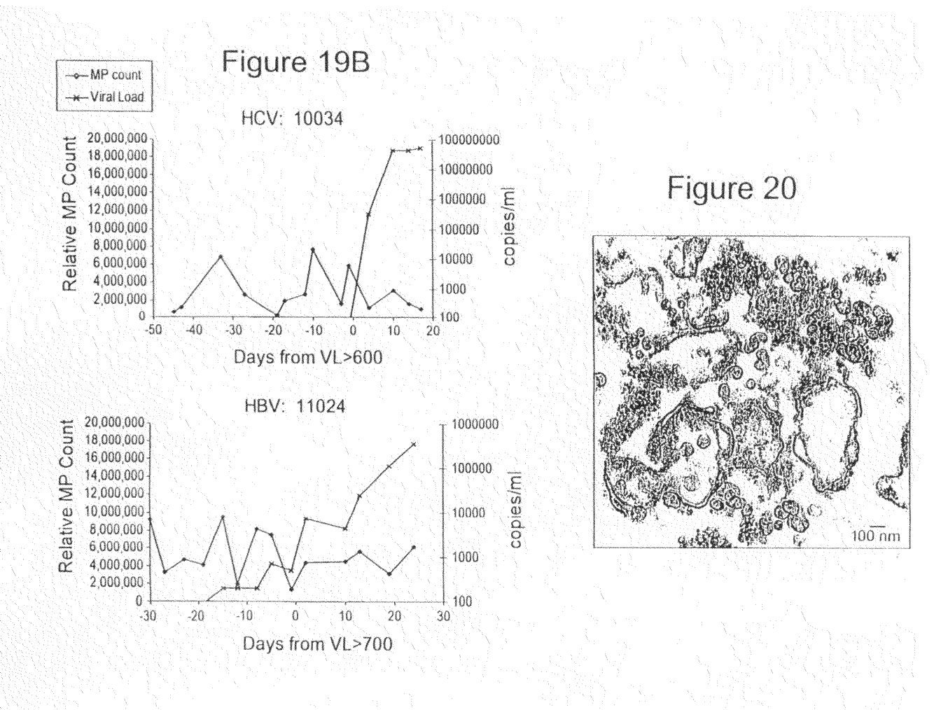

The present invention includes strategies to prevent apoptosis that include, but are not limited to, the use of PS-containing HIV immunogens, such as PS liposomes, either with or without CON-S or CON-T gp140 or HIV env epitopes associated with the liposomes, such as 2F5-GTH1 peptide lipid conjugates (FIGS. 8A and 8B) administered with adjuvants to break tolerance and induce anti-PS antibodies that inhibit PS-CD36 interactions. Alternatively, recombinant CD36 can be targeted in order to raise anti-CD36 antibodies, preferably, both anti-PS or anti-CD36 antibodies are induced at mucosal sites to prevent apoptotic mediated immune suppression.

Other strategies of the invention that can be used to preventapoptosis are inclusion of the HIV tat gene or protein in the HIV vaccine immunogen to induce antibodies against the tat protein that will inhibit the ability of tat to induce apoptosis in immune cells (Eusoli et Microbes Infect. 7:1392-9 (2005)). Forms of tat that can be used include the 101 amino acid tat protein or the gene encoding such a protein (Watkins et al, Retrovirology 3:1742 (2006)).

In addition to the above, a pancaspase inhibitor (e.g., zVAD-FMK (see also Dean et al. Cancer Treat. Rev. 33:203-212 (2007), Meng et al Current. Opinion Cell Biol. 18:668-676 (2006)) can be included in the vaccine to simultaneously inhibit any vaccine or immune cell activation associated with apoptosis to allow antibody responses to occur quickly. Any Env associated immunosuppression would be overcome. A pancaspase inhibitor can also be used to treat chronic HIV infection.

Correction of the immunosuppressive apoptotic insult can also be effected by immunizing with HIV antigens with various inhibitors of TNF such as Etanercept (a dimeric human TNFR p75-FC fusion protein) or with antibodies against TNFα (such as Infliximab or Adalimumab) (see “Rheumatoid Arthritis”, by EW St. Clair, DS Pisetsky and BF Haynes, Lippincott Williams and Wilkins, 2004, particularly chapters 31 and 32.) and an inhibitor of Fas-Fas ligand interactions (like Fas-Fc) and an inhibitor of TRAIL-DR5 interactions (such as DR5-Fc) (these can be used together or separately). Such agents can also be used to treat chronic HIV infection.

The components of the multicomponent vaccine of the invention can be formulated, as appropriate, with a pharmaceutically acceptable carrier using techniques well known in the art. Suitable routes of administration of the vaccine components include, as appropriate, systemic (e.g., intramuscular or subcutaneous), mucosal or intranasal. Optimum dosing regimens can be determined by one skilled in the art and can vary with the patient and specific components used.

Certain aspects of the invention can be described in greater detail in the non-limiting Examples that follows.

### Example 1

### Vaccine Components

The basic components of the multicomponent vaccine are:

1. a strategy to break immune tolerance,

2. an immunogen to overcome diversity and induce broadly reactive neutralizing antibodies,

3. a strategy to evade the immunosuppression associated with massive apoptosis of immune and other cells that occurs at the time of acute HIV infection,

4. a vector/formulation that provides mucosal immune responses.

An example of the invention is the following multicomponent immunogen:

DNA prime containing recombinant CON-S consensus gp160 HIV Env with a ICK cytoplasmic domain motif (break tolerance and deal with diversity, neutralizing antibody responses) recombinant boost with recombinant vesicular stomatitis virus containing CON-S gp140 Env and mosaic gag-nef genes, consensus pol, tat genes (deal with diversity, mucosal immune responses) recombinant CON-S gp140 protein prime and boost in type “B” or “C” oCpGs in a squalene emulsion administered with the DNA and rVSV immunizations (neutralizing antibody responses, break immune tolerance) combined with CD40-ligand and GITRL in a DNA plasmid administered with each immunization.

### Example 2

### Non-Human Primate Anthrax PA Vaccination Model (Rhesus)

A Rhesus T Reg cell depletion model has been developed to test the impact of transient T reg inactivation on the host immune response to anthrax protective antigen (rPA). ONTAK (15 mcg/Kg) infused for 5 days into rhesus monkeys significantly reduced (p<0.05) the percent of CD4+/CD25+ cells in peripheral blood (red line vs heavy black; FIG. 10). It is critical that the NHP (Rhesus) CD25 be monitored with the anti-huCD25 mAb clone 2A3 (BD Biosciences). It is also important to note that ONTAK is hulL-2-diptheria toxin and is known to delete CD25+ cells from the animal.

To test the hypothesis that ONTAK would improve the host immune response to a biodefense immunogen, juvenile Chinese rhesus monkeys were immunized with rPA (protective antigen; 25 μg) alone or in combination with 5 consecutive days of ONTAK (15 mcg/kg IV) infusion. Animals (n=3/group) were bleed for CBC/diff, immunophenotype, chemistry panel, plasma and serum on days—7, 5, 10, 12, 19, 33, 40 post immunization. Shown in FIG. 11 is the frequency of CD4+/CD25 T Reg cells in PB in the immunized groups. ONTAK infused monkeys have a distinct reduction in the T Reg cell compartment. The T Reg compartment in saline infused animals immunized with PA+Alum was not impacted.

Two measures were used to assess the magnitude and quality of the primary humoral response to PA in the NHP model +/−ONTAK. First, antigen-specific Ig isotype binding was studied and second, a determination was made of the ability of sera to neutralize anthrax toxin (PA+LF) in a TNA assay. The dose of PA (25 μg+Alum) used induced a anti-PA humoral response starting on day 19, as indicated by the geometric mean endpoint titer plotted on a log scale (FIG. 12). It was observed that ONTAK modestly improved the endpoint binding titer of PA-specific IgG and IgM following a single immunization on day 19, but this differential was not sustained out to day 40 (FIG. 12).

An anthrax toxin Neutralization Assay (TNA) has been established for use with mouse and rhesus serum. Test sera were run as a dilution series in the assay. Shown in FIG. 13A are the % neutralization curves for the optimal dilution of 1:512 over time. Shown in FIG. 13B is the NT₅₀ for the experimental groups at days 19, 33 and 40 post immunization. An improvement was observed with ONTAK versus PA+Alum alone in the peak anthrax toxin neutralizing titer 33 days post immunization, thus suggesting a functional enhancement of anti-PA responses with ONTAK in NHPs.

### Example 3

### Levels of Plasma FAS Ligand, TNTR2, TRAIL, and Apoptotic Microparticles are Elevated During Viral Load Ramp-Up in Acute HIV-1 Infection

### Experimental Details

### Plasma Samples

Seroconversion panels (HIV-1+/HCV-/HBV-, n=30, HIV-1-/HCV-/HBV+, n=10, and HIV-1-, HCV+/HCV-, n=10) were obtained from ZeptoMetrix Corporation, (Buffalo, N.Y.). Each panel consisted of sequential aliquots of plasma (range 4-30) collected approximately every 3 days from a plasma donor. HIV-1-/HCV-/HBV-human plasmas (n=25) were obtained from Innovative Research, (Southfield, Mich.). All studies were approved by the Duke University human subjects institutional review board.

### Viral Load Testing

Viral load testing of the plasma samples was performed by Quest Diagnostics (Lyndhurst, N.J.) RNA PCR Ultra). HCV and HBV viral loads were preformed by Zeptometrix: select HCV viral loads were provided by Philip Norris, Blood Systems Research Institute. San Francisco, Calif.

### ELISAs for Plasma Markers of Apoptosis

ELISAs for Fas, Fas Ligand, TRAIL (Diaclone, Besancon Cedex, France), and TNFR2 (Hycult Biotechnology, Uden, The Netherlands) were performed according to the manufacturer's directions. Plasma was assayed undiluted (TRAIL), diluted 1:10 (TNFR2) or diluted 1:2 (Fas Ligand).

### Apoptotic Microparticle (MP) Quantification

The number of MP in each plasma sample was determined with flow cytometry. All flow cytometry analyses were performed on the LSRII Flow Cytometer (BD Biosciences, San Jose, Calif.) and data analyses were performed using FlowJo software (Ashland, Oreg.). All buffers (PBS without calcium and magnesium) (Cellgro, Herndon, Va.) and formaldehyde (Sigma, St. Louis, Mo.) were filtered with a 0.22 am filter (Millipore, Billerica, Mass.) before use in any MP experiment. The buffer used to dilute plasma samples (1% formaldehyde in PBS without calcium and magnesium) was used to define the background MP count (˜1500 events counted in 60 seconds on the flow cytometer). To define the MP gate, FluoSpheres Fluorescent Microspheres (Molecular Probes, Eugene, Oreg.), ranging in size from 0.1 μm to 1 μm, were analyzed on the flow cytometer. The MP gate was drawn around the beads, encompassing the 0.1 μm, 0.2 μm, 0.5 μm, and 1.0 μm beads. Each plasma sample was diluted 1:100 and 1:1000 in 1% formaldehyde/PBS, and data acquired for 60 seconds. Optimal sample dilutions were determined experimentally, with the acceptance criteria being the dilution of plasma with abort counts <5%, and noise to signal ratios <0.1 (noise to signal ratio=background MP count in PBS/experimental plasma MP count) (FIGS. 14 and 15).

### Microparticle Phenotypic Analysis

Plasma samples (2 ml) were diluted in 5 ml of filtered saline and then filtered through a 5 μm filter (Pall Corporation, East Hills, N.Y.). The diluted samples were then centrifuged (1 hr at 200,000×g at 4° C.) (Sorvall RC M150 GX, Thermo Fisher Scientific, Waltham, Mass.). The top 2.5 ml of supernatant was removed, 2.5 ml of fresh saline added and samples were centrifuged×1 hr, 200,000×g. The pellet was washed ×2 in 1 ml of filtered saline; after the last wash, 900 μl of the supernatant was removed and the pellet resuspended in the remaining 200 μl of saline. Ten μl of MP suspension was incubated with an antibody and/or annexin V (total volume of 100 μl×20 minutes, 20° C., in the dark). Saline with 1% BSA (Sigma) was used as staining buffer for incubation with antibodies, and 2.5 mM CaCl₂ added to the buffer for annexin V staining. For annexin V control, 50 mM EDTA was added to the buffer, incubated 20 min., the volume adjusted to 500 μl with saline/formaldehyde, and analyzed by flow cytometry within 24 hours. Conjugated antibodies included mouse anti-human CD45-PE, CD3-PE, CD4-PE, CD6a, CD63, CCR5-PE, CD14-PE, CD19-PE, and isotype controls (BD Biosciences, San Jose, Calif.), and annexin V conjugated to AlexaFluor 647 (Molecular Probes, Eugene, Oreg.).

### Electron Microscopy of Plasma Microparticles

Eight ml of plasma was diluted 1:5 in filtered saline and MP pelleted (200,000×g×1 hr, 4° C.). Pellets were washed (200,000×g×1 hr, 4° C.). The pellet was resuspended in 1 ml of saline and washed ×2 (100,000×g×30 minutes). The MP pellet was resuspended in 500 μl of saline and overlaid onto 1 ml of a 40% sucrose solution, and MP centrifuged (100,000×g×90 min.). The pellets were fixed (1% formaldehyde, 4° C. overnight), pelleted, (100,00×g×60 min.), soaked in 1% osmium tetroxide×10 min. and rinsed with saline. The pellets were mounted in agar and embedded in epoxy resin and baked overnight at 60° C. Ultrathin sections were cut and stained and were examined with a Philips CM12 transmission electron microscope,

### Statistical Analyses

To establish a reference point throughout all the plasma seroconversion panels, Day “0” was defined as the date when viral load reached 100 copies/ml for HIV-1, 600 copies/ml for HCV, and 700 copies/ml for HBV.

To determine the percent increase in plasma markers of apoptosis during HIV-1, HBV, and HCV infections, the mean TRAIL, TNFR2, or Fas Ligand level before Day 0 was compared to the mean level after Day 0, and percent increase was calculated, ([(mean after day 0−mean before day 0)/mean after day 0]×100).

To compare the plasma markers of apoptosis during the course of infection, the mean levels of TRAIL. TNFR2, and Fas Ligand in uninfected donors, in the first sample of the seroconversion panel (first observation), and at the peak of viral load were compared in HIV-1 infection and in HBV and HCV infections (data not shown). Boxplot analyses were then performed for each group of data. Briefly, for each of the three groups compared, the maximum value, the minimum value, the mean value, and the first and third quartiles (encompassed by box) were calculated. Outliers (1.5× the difference between the third quartile and the first quartile of data) were omitted. Using a Students' t test, the means of each group were compared, and P values calculated.

To analyze the timing of the appearance of the plasma markers of apoptosis during HIV-1 infection, metrics were developed to characterize viral expansion rates. Metrics developed included maximum viral expansion rate, (r₀), and date of peak for each plasma marker of apoptosis. For these analyses, six subjects of the thirty total were excluded because the associated viral load data was too sparsely sampled to yield reliable metrics. Viral expansion rate (r₀) was determined using the two points within viral ramp-up which yield maximum expansion. For purposes of establishing the timing relationships between viral load and analyte metrics, Wilcoxon Rank Sum tests were performed for paired data. Each test performed compared date of maxium viral expansion with the date of a peak metric.

To optimize existing flow cytometric protocols for the investigations of microparticles, variety of experiments were performed. First, dilution series of polystyrene beads were assayed with the LSRII to determine acceptable signal to noise ratios and abort counts (FIG. 14). It was also determined experimentally that the expression levels of extracellular markers, such as CD3, CD45, the platelet marker CD61, and Annexin V decreased upon more than 1 freeze/thaw cycle, indicating the importance of sample integrity (FIG. 15).

### Results

TRAIL, TNFR2 and Fas Ligand were Elevated in Most Patients Either Just Before or During Viral Load Ramp-Up During Acute HIV-1 Infection.

To compare the viral kinetics, as well as the timing of the plasma markers of apoptosis and microparticle levels of one plasma donor patient to another, a common timepoint (Day 0) was determined for each of 30 HIV-1, 10 HCV and 10 HBV patients (FIG. 16). Day 0 was defined as the day that the patient's viral load reached 100 copies/ml, HCV viral load reached 600 copies/ml, and HBV viral load reached 700 copies/ml—levels that were imposed by the limits of detection for each viral load determination.

Next, to determine if changes of plasma markers of apoptosis could be detected at early timepoints in the acute HIV-1 infection process, levels of soluble TRAIL, TNFR2, and Fas Ligand were assayed in all plasma samples of each plasma donor that became HIV-1 viral load positive, and these levels were compared with those seen in HCV and HBV early infections, (FIG. 17). The percent change in plasma soluble TRAIL, TNFR2 and Fas Ligand levels were determined by comparing the mean analyte level before Day 0 to the mean after Day 0. Of the acute HIV-1 infected subjects, 27/30 demonstrated a greater than a 20% increase in TRAIL, 26/30 had increased TNFR2, and 23/30 had increased Fas Ligand levels. (FIG. 17B). In comparison, the HCV+ and HBV+ infected subjects demonstrated a >20% rise in TRAIL, TNFR2 or Fas Ligand only 0/10, 3/10, and 2/10 (HBV), in only 1/10, 6/10 and 7/10 subjects, respectively (HCV) (FIG. 17C).

Boxplot analyses were used to determine if analyte levels were significantly different at the time of peak viral load compared to samples drawn from the patient before viral load ramp up. The mean TRAIL, TNFR2, and Fas Ligand levels at the time of peak viral load, compared to the earliest plasma sample drawn from each acute HIV-1 infected patient before Day 0, were significantly different (p<0.01 for TRAIL, p<0.001 for TNRF2 and p<0.001 for Fas Ligand) (FIG. 18A). The peak TRAIL, TNFR2 and Fas Ligand levels were also significantly different from the levels of TRAIL, TNFR2, and Fas Ligand in uninfected plasma sample controls (p<0.001, p<0.001, and p<0.001, respectively) (FIG. 18A).

To investigate the timing of peak levels of TRAIL, TNFR2 and Fas Ligand compared to peak viral load, a determination was made of the relationship between the occurrence of an apoptotic analyte peak compared to the peak viral load, and the number of subjects that had peaks in plasma apoptotic analytes occurring before, coincident with or following the peak in HIV-1 viral load (Table 5). The majority of acute HIV-1 infection subjects (30/30 for TRAIL, 27/30 for TNFR2, and 26/30 for Fas Ligand), demonstrated peak analyte levels occurring within a 30-day time frame (i.e., 15 days before, at the time of, or within 15 days after the viral load peak) (Table 5). Of particular interest, the majority of subjects' TRAIL levels (21/30) peaked before the peak viral load, while TNFR2 and Fas Ligand levels more often peaked coincident with viral load (Table 5).

TABLE 5

| | | | | Peak | |
|---|---|---|---|---|---|
| | | Peak near | Peak before | coincident | Peak after |
| | | VL peak | VL | with VL | VL |
| HIV-1 | TRAIL | 30/30 | 21/30 | 9/30 | 0/30 |
| | TNFR2 | 27/30 | 7/30 | 16/30 | 4/30 |
| | Fas Ligand | 26/30 | 6/30 | 16/30 | 4/30 |
| HCV | TRAIL | 4/10 | 2/10 | 2/10 | 0/10 |
| | TNFR2 | 7/10 | 2/10 | 3/10 | 2/10 |
| | Fas Ligand | 6/10 | 4/10 | 1/10 | 1/10 |
| HBV | TRAIL | 5/10 | 4/10 | 1/10 | 0/10 |
| | TNFR2 | 4/10 | 2/10 | 2/10 | 0/10 |
| | Fas Ligand | 6/10 | 1/10 | 5/10 | 0/10 |

Within the 30 acute HIV-1 infected patients studied, the majority demonstrated TRAIL, TNFR2, and Fas Ligand level peaks near, (within 15 days), the peak viral load. Furthermore, the majority of patients demonstrated TRAIL level peaks before the viral load peaked, and TNFR2 and Fas Ligand level peaks coincident with the peak in viral load. The same analysis was performed for the 10 HCV and 10 HBV subjects studied.

To statistically analyze the timing of peak analyte levels relative to viral kinetics, paired Wilcoxon rank tests were performed (FIG. 18B). The significant p values indicate that the average day of peak analyte level was significantly different than the average day of peak viral expansion (r₀). Peak viral expansion rate indicates the date on which the virus is replicating at the maximum rate (mean day 5.5). Note that r₀ is distinct from Ro, the reproductive ratio. Importantly, these analyses demonstrated that TRAIL levels peaked first or 1.7 days after peak viral expansion. TNFR2 levels peaked next, 7.5 days after peak viral expansion, and Fas Ligand peaked 9.8 days after r₀. Analysis of the same panels reveals that the viral load reaches maximum levels at an average of 13.9 days after day 0 (median 13 days, interquartile range 3 days), indicating that TRAIL levels peak well before viral load peaks, while TNFR2 and Fas Ligand reached peak levels very close to the time of maximum viral load.

### Quantitative Flow Cytometry Analysis of Plasma Microparticles.

Because no concomitant peripheral blood mononuclear cell samples were available for the plasma panels, plasma panels were assayed for relative levels of plasma microparticles from ˜10 μM to 1.0 μM in size, and the presence of immune cell and exosome marker were determined on MP. Flow cytometry analyses were used to determine the relative levels of MP, comparing initial versus latency plasma samples from each individual (FIG. 19). To visualize plasma MP, transmission electron microscopy of MP banded on sucrose gradients was used. The relative number of MP present in each sample of the seroconversion panels was determined using the strategy outlined above (FIG. 14). A majority of acute HIV-1 infection subjects demonstrated peak MP numbers near (within 15 days before or 15 days after Day 0) the peak in viral load. Of the thirty HIV-1 seroconversion panels studied, 18 had peak microparticle numbers near the peak in viral load, and 11 of these 18 peaks occurred immediately before the peak in viral load, (Table 6). As controls, the HCV and HBV seroconversion panels were also analyzed to quantitate microparticle numbers, and no MP peaks were observed.

TABLE 6

| | HIV-1 | HCV | HBV |
|---|---|---|---|
| Peak MP Level Within 30 | 18/30 | 5/10 | 5/10 |
| days (+/−15) of Peak VL | | | |
| Peak MP Level Before Peak | 11/18 | 2/5 | 5/5 |
| VL | | | |
| Peak MP Level coincident with | 4/18 | 2/5 | 0/5 |
| Peak VL | | | |
| Peak MP Level after Peak VL | 3/18 | 1/5 | 0/5 |

Within the 30 acute HTV-1 infected patients studied, the majority demonstrated MP levels peaks near, (within 15 days), the peak in viral load, and a majority of those patients demonstrated MP peaks occurring before the peak in viral load.

### Phenotypic and Microscopic Analyes of Plasma Microparticles.

FIG. 20 is a transmission electron micrograph of plasma MPs following banding of MPs on sucrose gradients.

All documents and other information sources cited above are hereby incorporated in their entirety by reference.

1.-37. (canceled)

38. A method of inducing the production of an immune response against HIV-1 in a mammal comprising administering to said mammal:
i) a centralized HIV-1 gene sequence,
ii) an agent that breaks mammalian immune tolerance, and
iii) an agent that inhibits HIV-1-induced apoptosis or an immunosuppressive effect of HIV-1-induced apoptosis,
wherein (i), (ii) and (iii) are administered in amounts sufficient to effect said production.

39. The method according to claim 38 wherein said centralized HIV-1 gene sequence is a consensus or mosaic HIV-1 gene sequence.

40. The method according to claim 39 wherein said centralized HIV-1 gene sequence is present in a vector and wherein said vector is a viral vector or a recombinant mycobacterial vector.

41. The method according to claim 39 wherein said centralized HIV-1 gene sequence is a consensus HIV-1 gene sequence selected from the group consisting of a consensus HIV-1 env, gag, pol, nef and tat gene sequence or said centralized HIV-1 gene sequence is a mosaic HIV-1 gene sequence selected from the group consisting of a mosaic HIV-1 gag and nef gene sequence.

42. The method according to claim 38 wherein said centralized HIV-1 gene sequence comprises a sequence encoding a cytoplasmic domain endoplasmic reticulum retention sequence.

43. The method according to claim 38 wherein said agent that breaks mammalian immune tolerance is a T regulatory cell inhibitor or a TLR-9 agonist.

44. The method according to claim 43 wherein said agent that breaks mammalian immune tolerance comprises oligo CpGs.

45. The method according to claim 43 wherein said agent that breaks mammalian immune tolerance is a T regulatory cell inhibitor that comprises a glucocorticoid-induced TNF family-related receptor ligand (GITRL) encoding sequence, an anti-CD25 antibody or ONTAK.

46. The method according to claim 38 wherein said agent that inhibits HIV-1-induced apoptosis induces anti-phosphatidylserine (PS) antibodies, anti-CD36 antibodies, or anti-HIV tat antibodies.

47. The method according to claim 46 wherein agent that inhibits HIV-1-induced apoptosis induces anti-PS antibodies and comprises a PS liposome, wherein said PS-liposome comprises an HIV immunogen.

48. The method according to claim 46 wherein said agent that inhibits HIV-1-induced apoptosis induces anti-PS antibodies that inhibit PS-CD36 interactions or said agent that inhibits HIV-1-induced apoptosis induces anti-CD36 antibodies or said agent that inhibits HIV-1-induced apoptosis induces anti-HIV tat antibodies.

49. The method according to claim 38 wherein said agent that inhibits the immunosuppressive effect of HIV-1-induced apoptosis is administered and wherein said agent comprises a TNF inhibitor, wherein said TNF inhibitor comprises a monoclonal antibody against the TNF receptor, an inhibitor of Fas-Fas ligand interactions or an inhibitor of TRAIL-DR5 interactions.

50. The method according to claim 38 wherein said method further comprises administering to said mammal an amount of a pancaspase inhibitor sufficient to inhibit immune cell activation associated with HIV-1 induced-apoptosis.

51. The method according to claim 38 wherein said mammal is a human.

52. A composition comprising a centralized HIV-1 gene sequence, an agent that breaks mammalian immune tolerance and an agent that inhibits HIV-1-induced apoptosis or the immunosuppressive effect of apoptosis.
